(19) United States
(12) Patent Application Publication
(10) Pub. No.: US 2024/0352402 A1
(43) Pub. Date: Oct. 24, 2024

(54) ENGINEERED BACTERIA FOR BIOSYNTHESIS OF PEPTIDE-BASED QUORUM SENSING INHIBITORS

(71) Applicant: Wisconsin Alumni Research Foundation, Madison, WI (US)

(72) Inventors: Helen Elizabeth Blackwell, Middleton, WI (US); Danielle Lee Widner, Fitchburg, WI (US)

(73) Assignee: Wisconsin Alumni Research Foundation, Madison, WI (US)

(21) Appl. No.: 18/443,858

(22) Filed: Feb. 16, 2024

Related U.S. Application Data
(60) Provisional application No. 63/446,295, filed on Feb. 16, 2023.

(51) Int. Cl. C12N 1/20; C12N 15/75; C12R 1/125

(52) U.S. Cl. CPC C12N 1/205; C12N 15/75; C12N 2510/00; C12R 2001/125

(57) ABSTRACT

Described herein are engineered bacterial cells capable of producing an autoinducing peptide (AIP) or non-native AIP analog (including AIP inhibitors), methods for producing the AIP or non-native AIP analog, plasmids and kits. The bacterial cells are transformed with at least one plasmid into gram-positive bacterial cells expressing a mutation in the argD gene manipulating the AIP biosynthesis system via the AgrB/D to produce inhibitors of the S. aureus arg quorum sensing.

### CROSS-REFERENCE TO RELATED APPLICATIONS

This application claims the benefit of and priority to U.S. Patent Application No. 63/446,295, filed on Feb. 16, 2023, the contents of which are incorporated herein by reference in their entirety.

### GOVERNMENT SUPPORT

This invention was made with government support under 2108511 awarded by the National Science Foundation. The government has certain rights in the invention.

### SEQUENCE LISTING

The instant application contains a Sequence Listing which has been submitted electronically in XML file format and is hereby incorporated by reference in its entirety. Said XML, copy, created on Jun. 3, 2024, is named 032026-1526_SL.xml and is 69,153 bytes in size.

### TECHNICAL FIELD

Described herein, are engineered bacterial cells capable of producing an autoinducing peptide (AIP) or non-native AIP analog and methods for producing the same whereby the AIP and non-native AIP analogs inhibit the accessory gene regulator (agr) quorum sensing (QS) systems. Specifically, the present disclosure provides, among other things, engineered bacterial cells transformed with at least one plasmid, methods of producing AIP and non-native AIP analogs, plasmids, and kits comprising the same.

### BACKGROUND

The following description of the background of the present technology is provided simply as an aid in understanding the present technology and is not admitted to describe or constitute prior art to the present technology.

Quorum sensing (QS) is a form of bacterial cell-to-cell communication. It is the process by which bacteria of the same species coordinate behavior at high population density. When a continuously produced QS signal reaches a sufficient concentration, bacteria will turn on a set of genes specific to that QS signal. In many pathogenic bacteria, the genes activated are related to virulence, meaning compounds that disrupt QS could act as therapeutics. For example, the agr-type QS systems regulates virulence in many clinically relevant Gram-positive pathogens, including Staphylococcus aureus, Staphylococcus epidermidis, Listeria monocytogenes, and Clostridioides difficile.

Agr-type QS systems have four central components, AgrA-D. AgrD is the precursor peptide for the autoinducing peptide (AIP) signal, buildup of which triggers QS activation. AgrD contains an N-terminal leader sequence that localizes the peptide to the cell membrane for processing and transport, a central region that contains the final AIP product, and a charged C-terminal region that is essential for processing by AgrB. AgrB is a membrane bound endopeptidase that is responsible for removal of the C-terminal region and formation of the thiolactone moiety that gives AIP signals their distinct macrocyclic structure. Following processing by AgrB, the extracellular protease MroQ removes the N-terminal leader and—through an unknown mechanism—the AIP signal is transported across the cell membrane to diffuse freely into the environment. The AIP signal differs between species and even between strains. For example, S. aureus has four distinct QS specificity groups (I-IV), each with a unique AIP signal. Once the local extracellular AIP concentration is sufficiently high, the AIP ligand binds to the extracellular domain of the transmembrane receptor AgrC, triggering the phosphorylation of the response-regulator AgrA. AgrA is a DNA-binding protein that upregulates the agrBDCA operon and RNAIII, a regulatory RNA that in S. aureus and other pathogens controls expression of myriad virulence factors.

### SUMMARY OF THE INVENTION

The present technology manipulates the native AIP biosynthesis system via AgrB/D to biosynthesize inhibitors of the S. aureus accessory gene regulator (agr) quorum sensing (QS) system demonstrating the ability of strains that produce non-native AIP analogs to antagonize S. aureus QS in a mixed-microbial environment. In one aspect, the present disclosure provides an engineered bacterial cell capable of producing an autoinducing peptide (AIP) or non-native AIP analog, wherein the engineered bacterial cell is transformed with at least one plasmid comprising one or more nucleotide sequences encoding one or more of agrBD loci, mroQ gene, argB gene, or argD gene, and one or more promoters. In another aspect, the present disclosure provides an engineered bacterial cell capable of producing a non-native autoinducing peptide (AIP) analog that inhibits the accessory gene regulator (agr) quorum sensing (QS) system, wherein the engineered bacterial cell is transformed with at least one plasmid comprising one or more nucleotide sequences encoding one or more of agrBD loci, mroQ gene, argB gene, or argD gene, and one or more promoters.

In any embodiments, the engineered bacterial cell is an engineered Gram-positive bacterium.

In any embodiments, the Gram-positive bacterium is selected from B. subtilis strain or P. megaterium strain.

In any embodiments, the B. subtilis strain is Bs-ΔydiL.

In any embodiments, the one or more promoters are selected from the group consisting of PliaG promoter, PlepA promoter, and Pveg promoter.

In any embodiments, the plasmid comprises at least the nucleotide sequence encoding the PliaG promoter and the mroQ gene.

In any embodiments, the engineered bacterial cell comprises a second plasmid comprising a nucleotide sequence encoding at least the agrBD loci.

In any embodiments, the argBD loci is amplified from wild type S. aureus.

In any embodiments, the wild type S. aureus comprises a group I or group III type arg QS system.

In any embodiments, the plasmid further comprises a ribosome-binding sequence of AAGGAGG and one or more of 7-nucleotide spacer inserted 5′ of each coding gene.

In any embodiments, the nucleotide sequence 3′ of the argD gene further comprises a terminator stem sequence.

In any embodiments, the engineered bacterial cell comprises at least one mutation in the argD gene.

In any embodiments, the mutation in the argD gene is D29A.

In any embodiments, the argD gene is mutated and encoded by a nucleic acid sequence selected from SEQ ID NOs: 41 or 42.

In any embodiments, the bacterial cell is an inducible bacterial strain or a constitutive strain.

In any embodiments, the agr QS system is an S. aureus agr QS system.

In another aspect, the present disclosure provides an engineered bacterial cell transformed with one or more of: (a) a plasmid comprising a nucleic acid sequence encoding a agrBD locus amplified from wild type S. aureus; (b) a first plasmid comprising a nucleic acid sequence encoding a agrBD locus amplified from wild type S. aureus and a second plasmid comprising a nucleic acid sequence encoding a PliaG promoter and a mroQ gene; or (c) a plasmid comprising a nucleic acid sequence encoding (i) a promoter selected from PliaG promoter, PlepA promoter, or Pveg promoter; (ii) a mroQ gene, an argB gene, and an argD gene; and (iii) a ribosome-binding sequence of AAGGAGG and one or more of 7-nucleotide spacer inserted 5′ of each coding gene.

In any embodiments, the argD gene is mutated and encoded by a nucleic acid sequence selected from SEQ ID NO: 41 or 42.

In any embodiments, the mutation is incorporated into the plasmid by site-directed mutagenesis.

In one aspect, the present disclosure provides a plasmid comprising one or more of the nucleic acid sequences as disclosed herein.

In another aspect, the present disclosure provides a method of producing an autoinducing peptide (AIP) or non-native AIP analog comprising, transforming a bacterial cell with one or more of the plasmids as disclosed herein.

In another aspect, the present disclosure provides a method of producing a non-native autoinducing peptide (AIP) analog comprising, transforming a bacterial cell with one or more of: (a) a plasmid comprising a nucleic acid sequence encoding a agrBD locus amplified from wild type S. aureus; (b) a first plasmid comprising a nucleic acid sequence encoding a agrBD locus amplified from wild type S. aureus and a second plasmid comprising a nucleic acid sequence encoding a PliaG promoter and a mroQ gene; or (c) a plasmid comprising a nucleic acid sequence encoding: (i) a promoter selected from PliaG promoter, PlepA promoter, or Pveg promoter; (ii) a mroQ gene, an argB gene, and a mutated argD gene; and (iii) a ribosome-binding sequence of AAGGAGG and one or more of 7-nucleotide spacer inserted 5′ of each coding gene.

In any embodiments, the AIP analog is an inhibitor of S. aureus agr quorum sensing (QS) system.

In one aspect, the present disclosure provides kits comprising one or more plasmids, and/or the engineered bacterial cell as disclosed herein and optionally, instructions for use.

In any embodiments, the kit comprises one or more primers selected from the group consisting of SEQ ID NOs: 1-27.

The foregoing general description and following detailed description are exemplary and explanatory and are intended to provide further explanation of the disclosure as claimed. Other objects, advantages, and novel features will be readily apparent to those skilled in the art from the following brief description of the drawings and detailed description of the disclosure.

### BRIEF DESCRIPTION OF THE DRAWINGS

FIG. 1: depicts a schematic for generating strains of B. subtilis that inducibly produce AIPs and AIP analogs.

FIG. 2: depicts the results of inhibition curves for AIP-I D5A (strain Pm-G1D5A) (left) and AIP-III D4A (strain Pm-G3D4A) (right) from P. megaterium supernatant. P. megaterium supernatant was lyophilized and added to a reporter assay measuring agr activity. High concentrations of AIP analog containing supernatant inhibit agr. Wild type (WT) P. megaterium supernatant was used as a vehicle control. Assays were performed against each of the four agr specificity groups (Groups I, II, III, and IV) (n=3).

FIG. 3: depicts the results of inhibition curves for AIP-I D5A (strain Bs-G1D5A) (left) and AIP-III D4A (strain Bs-G3D4A) (right) from B. subtilis supernatant. B. subtilis supernatant was lyophilized and added to a reporter assay measuring agr activity. High concentrations of AIP analog containing supernatant inhibit agr. Wild type (WT) B. subtilis supernatant was used as a vehicle control. Assays were performed against each of the four agr specificity groups (Groups I, II, III, and IV) (n=9).

FIG. 4: depicts genetic modules for constitutive expression of AIP-I D5A and AIP-III D4A. P_{liaG}, P_{lepA}, and P_{veg} are constitutive B. subtilis promoters of intermediate, strong, and very strong strength, respectively. The mroQ, agrB, and agrD genes were amplified from S. aureus genomic DNA. Site-directed mutagenesis was performed on the agrD genes to create the desired non-native AIP analog. Modules were constructed using BioBrick cloning technique.

FIG. 5: depicts the results of inhibition of S. aureus AgrC-I, AgrC-II, AgrC-III and AgrC-IV by supernatant from B. subtilis strains that constitutively produce AIP-I D5A and AIP-III D4A. Values are reported as the percentage of B. subtilis supernatant required to reach 50% inhibition. Samples where an IC₅₀ value could not be calculated are reported as >100. (n=9).

FIG. 6: depicts results of competition assays with various strains of B. subtilis and S. aureus. Values for 100 percent activation were calculated by averaging normalized fluorescence readings for three samples of S. aureus without B. subtilis (n=9).

### DETAILED DESCRIPTION

### Definitions

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

The following terms are used throughout as defined below. All other terms and phrases used herein have their ordinary meanings as one of skill in the art would understand.

The practice of the present technology will employ, unless otherwise indicated, conventional techniques of tissue culture, molecular biology, microbiology, chemical engineering, and cell biology, which are within the skill of the art.

Unless the context indicates otherwise, it is specifically intended that the various features described herein can be used in any combination. Moreover, the disclosure also contemplates that in some embodiments, any feature or combination of features set forth herein can be excluded or omitted. To illustrate, if the specification states that a complex comprises components A, B, and C (or A, B, and/or C), it is specifically intended that any of A, B or C, or a combination thereof, can be omitted and disclaimed singularly or in any combination.

All numerical designations, e.g., pH, temperature, time, concentration, and molecular weight, including ranges, are approximations which are varied (+) or (−) by increments of 1.0 or 0.1, as appropriate, or alternatively by a variation of +/−10%, or alternatively 5%, or alternatively 2%. It also is to be understood, although not always explicitly stated, that the reagents described herein are merely exemplary and that equivalents of such are known in the art.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context.

Also as used herein, “and/or” refers to and encompasses any and all possible combinations of one or more of the associated listed items, as well as the lack of combinations when interpreted in the alternative (“or”).

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The terms or “acceptable,” “effective,” or “sufficient” when used to describe the selection of any components, ranges, dose forms, etc. disclosed herein intend that said component, range, dose form, etc. is suitable for the disclosed purpose.

As used herein, the terms “engineered bacterial cell” refers to a modified bacterial cell. Non-limiting examples of modifications include enhanced expression of a gene, inhibited expression of a gene, introduction of new gene(s), introduction of mutant gene(s), or mutation of gene(s), wherein the enhanced expression or inhibited expression of a gene can be achieved by using common techniques in the art, such as gene deletion, changed gene copy number, introduction of a plasmid, changed gene promoter (e.g. by using a strong or weak promoter).

As used herein, the term “strain” refers to a bacteria of a particular species which have common characteristics. Unless indicated to the contrary, the terms “strain” and “cell” are used interchangeably herein. Bacterial strains are composed of individual bacterial cells and individual bacterial cells have specific characteristics. Non-limiting examples include a particular growth rate or level of target biomolecule production which identifies them as being members of their particular strain.

As used herein, the term “Gram-positive bacteria” refers to bacteria that give a positive gram stain test result. Gram-positive bacteria take up crystal violet stain due to their thick peptidoglycan layer in the bacterial cell wall.

As used herein, the term “Gram stain test” or “Gram stain” refers to a method of staining used to classify bacterial species into two broad group such as Gram-positive bacteria and Gram-negative bacteria.

As used herein, the term “promoter” refers to a DNA sequence capable of controlling the expression of a coding sequence or functional RNA. In some embodiments, the promoter sequence consists of proximal and more distal upstream elements, the latter elements often referred to as enhancers. Accordingly, an “enhancer” is a DNA sequence that can stimulate promoter activity, and may be an innate element of the promoter or a heterologous element inserted to enhance the level or tissue specificity of a promoter. Promoters may be derived in their entirety from a native gene, or be composed of different elements derived from different promoters found in nature, or even comprise synthetic DNA segments. Different promoters may direct the expression of a gene in different tissues or cell types, or at different stages of development, or in response to different environmental conditions. Since in most cases the exact boundaries of regulatory sequences have not been completely defined, DNA fragments of some variation may have identical promoter activity.

As used herein, the term “mutation” refers to an insertion, deletion or substitution in a nucleic acid molecule. When present in the coding region of a nucleic acid, a mutation maybe “silent” (i.e., results in no phenotypic effect) or may alter the function of the expression product of the coding region. When a mutation occurs to the regulatory region of a gene or operon, the mutation may either have no effect or alter the expression characteristics of the regulated nucleic acid.

As used herein, the term “site directed mutagenesis” refers to creating specific targeted changes in double stranded plasmid DNA. Non-limiting examples include insertions, deletions, and substitutions.

As used herein, the terms “terminator stem sequence” is a nucleotide sequence that determines the detachment of RNA polymerase from the DNA template strand and signals the end of transcription.

As used herein, the terms “nucleic acid sequence,” “nucleic acid molecule,” or “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising, or alternatively consisting essentially of, or yet further consisting of purine and pyrimidine bases or other natural, chemically or biochemically modified, non-natural, or derivatized nucleotide bases.

As used herein, the term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

### Engineered Bacterial Cell

The present technology provides engineered bacterial cells capable of producing an autoinducing peptide (AIP) or AIP analog, wherein the engineered bacterial cell is transformed with at least one plasmid comprising one or more nucleotide sequences encoding one or more of agrBD loci, mroQ gene, argB gene, or argD gene, and one or more promoters.

In certain embodiments, disclosed herein is an engineered bacterial cell capable of producing a non-native autoinducing peptide (AIP) analog that inhibits the accessory gene regulator (agr) quorum sensing (QS) system, wherein the engineered bacterial cell is transformed with at least one plasmid comprising one or more nucleotide sequences encoding one or more of agrBD loci, mroQ gene, argB gene, or argD gene, and one or more promoters.

In certain embodiments, disclosed herein is an engineered bacterial cell transformed with one or more of: (a) a plasmid comprising a nucleic acid sequence encoding a agrBD locus amplified from wild type S. aureus; (b) a first plasmid comprising a nucleic acid sequence encoding a agrBD locus amplified from wild type S. aureus and a second plasmid comprising a nucleic acid sequence encoding a PliaG promoter and a mroQ gene; or (c) a plasmid comprising a nucleic acid sequence encoding (i) a promoter selected from PliaG promoter, PlepA promoter, or Pveg promoter; (ii) a mroQ gene, an argB gene, and an argD gene; and (iii) a ribosome-binding sequence of AAGGAGG and one or more of 7-nucleotide spacer inserted 5′ of each coding gene.

In some embodiments, the agr QS system is an S. aureus agr QS system.

In some embodiments, the engineered bacterial cell is an engineered Gram-positive bacterium. In any embodiments, the Gram-positive bacterium is selected from B. subtilis strain or P. megaterium strain. In any embodiments, the Gram-positive bacterium is a B. subtilis strain. In any embodiments, the Gram-positive bacterium is P. megaterium.

In some embodiments, the engineered bacterial cell is a genetically traceable strain of B. subtilis or P. megaterium. In any embodiments, the engineered bacterial cell is from the phylum Bacillota. In any embodiments, the engineered bacterial cell is a Lactobacillusspecies. In any embodiments, the engineered bacterial cell is an Escherichia coli species.

The argBD loci can be amplified from any bacterial species that uses an agr system. In some embodiments, the argBD is amplified from wild type S. aureus. In any embodiments, the argBD is amplified from a species selected from Staphylococcus epidermidis, Listeria monocytogenes, or Clostridioides difficile. In any embodiments, the wild type S. aureus comprises a Group I or Group III agr system. In any embodiments, the argBD loci is an argBD loci Group I. In any embodiments, the argBD loci is an argBD loci Group III. In any embodiments, the argBD loci is encoded by a nucleic acid sequence having at least 55%, at least 65%, at least 70%, at least 75%, at least 80%, at least 85%, at least 90%, at least 95%, at least 97%, at least 98%, at least 99%, or 100% sequence identity to SEQ ID NO: 31 or 32. In any embodiments the argBD loci is encoded by the nucleic acid sequence of SEQ ID NO: 31. In any embodiments the argBD loci is encoded by the nucleic acid sequence of SEQ ID NO: 32.

In some embodiments, the mroQ gene is encoded by a nucleic acid sequence having at least 55%, at least 65%, at least 70%, at least 75%, at least 80%, at least 85%, at least 90%, at least 95%, at least 97%, at least 98%, at least 99%, or 100% sequence identity to SEQ ID NO: 36. In any embodiments, the mroQ is from S. aureus. In any embodiments, the P. megaterium strain has not been genetically modified to express S. aureus mroQ.

The ydiL gene is a homolog of mroQ. In some embodiments, the B. substilis strain is Bs-ΔydiL.

In some embodiments, the argB gene is selected from the argB gene Group I or the argB gene Group III. In any embodiments, the argB gene is argB gene Group I. In any embodiments, the argB gene is argB gene Group III. In any embodiments, the argB gene is encoded by a nucleic acid sequence having at least 55%, at least 65%, at least 70%, at least 75%, at least 80%, at least 85%, at least 90%, at least 95%, at least 97%, at least 98%, at least 99%, or 100% sequence identity to SEQ ID NO: 37 or 38. In any embodiments the argBD loci is encoded by the nucleic acid sequence of SEQ ID NO: 37. In any embodiments the argBD loci is encoded by the nucleic acid sequence of SEQ ID NO: 38.

In some embodiments, the argD gene is selected from the argD gene Group I or the argD gene Group III. In any embodiments, the argD gene is argD gene Group I. In any embodiments, the argD gene is argD gene Group III. In any embodiments, the argD gene is encoded by a nucleic acid sequence having at least 55%, at least 65%, at least 70%, at least 75%, at least 80%, at least 85%, at least 90%, at least 95%, at least 97%, at least 98%, at least 99%, or 100% sequence identity to SEQ ID NO: 39 or 40. In any embodiments, the argD gene is encoded by the nucleic acid sequence of SEQ ID NO: 39. In any embodiments the argD gene is encoded by the nucleic acid sequence of SEQ ID NO: 40. In any embodiments, the argD gene further comprises a terminator stem sequence. In any embodiments, the terminator sequence is 3′ of the argD gene. In any embodiments, the terminator stem sequence is encoded by a nucleic acid sequence having at least 55%, at least 65%, at least 70%, at least 75%, at least 80%, at least 85%, at least 90%, at least 95%, at least 97%, at least 98%, at least 99%, or 100% sequence identity to SEQ ID NO: 43.

In some embodiments, the engineered bacterial cell comprises at least one mutation in the argD gene. In any embodiments, the engineered bacterial cell comprises one or more mutations in the argD gene. In any embodiments, engineered bacterial cell comprises one or more, two or more, three or more, four or more, five or more, six or more, seven or more, eight or more, nine or more, or ten or more mutations in the argD gene. In any embodiments, the mutation is in the agrD gene Group I. In any embodiments, the mutation is on the argD gene Group III. In any embodiments, at least one mutation is the agrD_D29A Group I mutation. In any embodiments, at least one mutation is the agrD_D29A Group III mutation. In any embodiments, the agrD_D29A Group I mutation is encoded by a nucleic acid sequence having at least 55%, at least 65%, at least 70%, at least 75%, at least 80%, at least 85%, at least 90%, at least 95%, at least 97%, at least 98%, at least 99%, or 100% sequence identity to SEQ ID NO: 41. In any embodiments, the argD D4A Group III mutation is encoded by a nucleic acid sequence having at least 55%, at least 65%, at least 70%, at least 75%, at least 80%, at least 85%, at least 90%, at least 95%, at least 97%, at least 98%, at least 99%, or 100% sequence identity to SEQ ID NO: 42. In any embodiments, the at least one mutation is incorporated into the plasmid by site-directed mutagenesis.

In some embodiments, the engineered bacterial cell comprises one or more, two or more, three or more, four or more, or five or more promoters. In any embodiments, the engineered bacterial cell comprises two promoters. In any embodiments, each promoter is operably linked to a nucleic acid sequence that encodes a gene. In some embodiments, one or more promoters are selected from the group consisting of PliaG promoter, PlepA promoter, and Pveg promoter. In any embodiments, the promoter is PliaG promoter. In any embodiments, the PliaG-promoter is encoded by a nucleic acid sequence having at least 55%, at least 65%, at least 70%, at least 75%, at least 80%, at least 85%, at least 90%, at least 95%, at least 97%, at least 98%, at least 99%, or 100% sequence identity to SEQ ID NO: 33. In any embodiments, the PlepA-promoter is encoded by a nucleic acid sequence having at least 55%, at least 65%, at least 70%, at least 75%, at least 80%, at least 85%, at least 90%, at least 95%, at least 97%, at least 98%, at least 99%, or 100% sequence identity to SEQ ID NO: 34. In any embodiments, The Pveg promoter is encoded by a nucleic acid sequence having at least 55%, at least 65%, at least 70%, at least 75%, at least 80%, at least 85%, at least 90%, at least 95%, at least 97%, at least 98%, at least 99%, or 100% sequence identity to SEQ ID NO: 35.

In some embodiments, the engineered bacterial cell is an inducible bacterial strain. In any embodiments, the expression of genes is induced by xylose-inducible expression. In any embodiments, the engineered bacterial cell is a constitutive strain.

In some embodiments, the 7-nucleotide spacer can be any suitable spacer sequence. In any embodiments, the spacer sequence is CTGACTA.

### Plasmids

Embodiments described herein generally relate to one or more plasmids that are transformed into the disclosed bacterial cell. For example, the disclosed bacterial cell is transformed with one or more plasmids.

In certain embodiments, the plasmid comprises one or more nucleic acid sequence selected from the group consisting of SEQ ID NOs: 31-45.

In some embodiments, at least one plasmid comprises a nucleic acid sequence encoding the argBD loci. In any embodiments, the agrBD locus is amplified from wild type S. aureus. In some embodiments, the argBD loci is a argBD loci Group I. In some embodiments, the argBD loci is a argBD loci Group III. In any embodiments, the argBD loci is encoded by a nucleic acid sequence having at least 55%, at least 65%, at least 70%, at least 75%, at least 80%, at least 85%, at least 90%, at least 95%, at least 97%, at least 98%, at least 99%, or 100% sequence identity to SEQ ID NO: 31 or 32. In any embodiments the argBD loci is encoded by the nucleic acid sequence of SEQ ID NO: 31. In any embodiments the argBD loci is encoded by the nucleic acid sequence of SEQ ID NO: 32.

In some embodiments, at least one plasmid comprises a nucleic acid sequence encoding the PliaG promoter and the mroQ gene. In any embodiments, the PliaG-promoter is encoded by a nucleic acid sequence having at least 55%, at least 65%, at least 70%, at least 75%, at least 80%, at least 85%, at least 90%, at least 95%, at least 97%, at least 98%, at least 99%, or 100% sequence identity to SEQ ID NO: 33. In any embodiments, the mroQ gene is encoded by a nucleic acid sequence having at least 55%, at least 65%, at least 70%, at least 75%, at least 80%, at least 85%, at least 90%, at least 95%, at least 97%, at least 98%, at least 99%, or 100% sequence identity to SEQ ID NO: 36.

In some embodiments, at least one plasmid comprises a promoter, a mroQ gene, and argB gene, an argD gene, a ribosome-binding sequence of AAGGAGG and one or more spacers. In any embodiments, the argD comprises a mutation. In any embodiments, the promotor is selected from PliaG promoter, PlepA promoter, or Pveg promoter. In any embodiments, the promoter is PliaG promoter. In any embodiments, the PliaG-promoter is encoded by a nucleic acid sequence having at least 55%, at least 65%, at least 70%, at least 75%, at least 80%, at least 85%, at least 90%, at least 95%, at least 97%, at least 98%, at least 99%, or 100% sequence identity to SEQ ID NO: 33. In any embodiments, the promoter is the PlepA-promoter. In any embodiments, the PlepA-promoter is encoded by a nucleic acid sequence having at least 55%, at least 65%, at least 70%, at least 75%, at least 80%, at least 85%, at least 90%, at least 95%, at least 97%, at least 98%, at least 99%, or 100% sequence identity to SEQ ID NO: 34. In any embodiments, the promoter is Pveg. In any embodiments, the Pveg promoter is encoded by a nucleic acid sequence having at least 55%, at least 65%, at least 70%, at least 75%, at least 80%, at least 85%, at least 90%, at least 95%, at least 97%, at least 98%, at least 99%, or 100% sequence identity to SEQ ID NO: 35. In any embodiments, the mroQ gene is encoded by a nucleic acid sequence having at least 55%, at least 65%, at least 70%, at least 75%, at least 80%, at least 85%, at least 90%, at least 95%, at least 97%, at least 98%, at least 99%, or 100% sequence identity to SEQ ID NO: 36. In any embodiments, the argB gene is selected from the argB gene Group I or the argB gene Group III. In any embodiments, the argB gene is argB gene Group I. In any embodiments, the argB gene is argB gene Group III. In any embodiments, the argB gene is encoded by a nucleic acid sequence having at least 55%, at least 65%, at least 70%, at least 75%, at least 80%, at least 85%, at least 90%, at least 95%, at least 97%, at least 98%, at least 99%, or 100% sequence identity to SEQ ID NO: 37 or 38. In any embodiments the argB gene is encoded by the nucleic acid sequence of SEQ ID NO: 37. In any embodiments the argB gene is encoded by the nucleic acid sequence of SEQ ID NO: 38. In any embodiments, the argD gene is selected from the argD gene Group I or the argD gene Group III. In any embodiments, the argD gene is argD gene Group I. In any embodiments, the argD gene is argD gene Group III. In any embodiments, the argD gene is encoded by a nucleic acid sequence having at least 55%, at least 65%, at least 70%, at least 75%, at least 80%, at least 85%, at least 90%, at least 95%, at least 97%, at least 98%, at least 99%, or 100% sequence identity to SEQ ID NO: 39 or 40. In any embodiments, the argD gene is encoded by the nucleic acid sequence of SEQ ID NO: 39. In any embodiments the argD gene is encoded by the nucleic acid sequence of SEQ ID NO: 40. In any embodiments, the argD gene further comprises a terminator stem sequence. In any embodiments, the terminator sequence is 3′ of the argD gene. In any embodiments, the terminator stem sequence is encoded by a nucleic acid sequence having at least 55%, at least 65%, at least 70%, at least 75%, at least 80%, at least 85%, at least 90%, at least 95%, at least 97%, at least 98%, at least 99%, or 100% sequence identity to SEQ ID NO: 43. In any embodiments, one or more spacers are 7-nucleotides in length. In any embodiments, at least one spacer is CTGACTA

In some embodiments, at least one mutation is incorporated into at least one plasmid. In any embodiments, the at least one mutation is incorporated into the plasmid by site-directed mutagenesis. In any embodiments, one mutation is the agrD_D29A Group I mutation. In any embodiments, at least one mutation is the agrD_D4A Group III mutation. In any embodiments, the agrD_D29A Group I mutation is encoded by a nucleic acid sequence having at least 55%, at least 65%, at least 70%, at least 75%, at least 80%, at least 85%, at least 90%, at least 95%, at least 97%, at least 98%, at least 99%, or 100% sequence identity to SEQ ID NO: 41. In any embodiments, the argD D4A Group III mutation is encoded by a nucleic acid sequence having at least 55%, at least 65%, at least 70%, at least 75%, at least 80%, at least 85%, at least 90%, at least 95%, at least 97%, at least 98%, at least 99%, or 100% sequence identity to SEQ ID NO: 42.

In some embodiments, one or more plasmids comprise nucleic acids sequence SEQ ID NOs: 28-30. In any embodiments, one or more plasmids are selected from the plasmids in Table 2.

### Methods for Producing AIP and AIP Analog

Disclosed herein, are methods for producing an autoinducing peptide (AIP) or AIP analog comprising transforming a bacterial cell with one or more plasmids as disclosed herein.

In certain embodiments, disclosed herein are methods of producing a non-native autoinducing peptide (AIP) analog comprising, transforming a bacterial cell with one or more of: (a) a plasmid comprising a nucleic acid sequence encoding a agrBD locus amplified from wild type S. aureus; (b) a first plasmid comprising a nucleic acid sequence encoding a agrBD locus amplified from wild type S. aureus and a second plasmid comprising a nucleic acid sequence encoding a P_{liaG} promoter and a mroQ gene; or (c) a plasmid comprising a nucleic acid sequence encoding: (i) a promoter selected from P_{liaG} promoter, P_{lepA} promoter, or P_{veg} promoter; (ii) a mroQ gene, an argB gene, and a mutated argD gene; and (iii) a ribosome-binding sequence of AAGGAGG and one or more of 7-nucleotide spacer inserted 5′ of each coding gene.

In any embodiments, a bacterial cell is an engineered bacterial cell.

In any embodiments, a bacterial cell is transformed with at least one plasmid comprising a nucleic acid sequence encoding a agrBD locus as disclosed herein. In any embodiments, a bacterial cell is transformed with at least a first plasmid comprising a nucleic acid sequence encoding a agrBD locus as described herein and at least a second plasmid comprising a nucleic acid sequence encoding a P_{liaG} promoter and a mroQ gene as disclosed herein. In any embodiments, a bacterial cell is transformed with at least one plasmid encoding a promoter, a mroQ gene, an argB gene, and an argD gene as disclosed herein. In any embodiments, a bacterial cell is transformed with at least one plasmid that comprises a ribosome-binding sequence of AAGGAGG and one or more 7-nucleotide spacers inserted 5′ of each gene as disclosed herein. In any embodiments, at least one plasmid has a argD mutation incorporated by site directed mutagenesis.

In any embodiments, the AIP analog is an inhibitor of S. aureus agr quorum sensing.

### Pharmaceutical Compositions and Kits

In some aspects, the disclosed engineered bacterial cell, the produced AIP, the produced non-native AIP analog or a combination thereof is provided in a pharmaceutical composition. In any embodiments, the composition comprises the engineered bacterial strain, the produced AIP, the produced non-native AIP analog or a combination thereof and a pharmaceutically acceptable carrier, excipient, and/or diluent. Examples of suitable carriers, excipients, and diluents include lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, minerals, and the like. In some embodiments, the engineered bacteria cell is in water or another pharmaceutically acceptable aqueous carrier in which the conjugate exhibits good solubility, optionally with or without other pharmaceutical acceptable excipients, or preservatives.

Also provided herein are kits. A kit may comprises one or more of the plasmids and one or more of the engineered bacterial cells disclosed herein, contained in a suitable container, optional together with instructions for use in a method as disclosed herein. In any embodiments, the kit further comprising one or more primers as disclosed herein. In any embodiments, the primers are selected from the group consisting of SEQ ID NOs: 1-27. In any embodiments, the primers comprises at least one forward and at least one reverse primer.

### EXAMPLES

These examples are provided for illustrative purposes only and not to limit the scope of the claims provided herein. The examples herein are provided to illustrate advantages of the present technology and to further assist a person of ordinary skill in the art with preparing or using the compositions and systems of the present technology. The examples should in no way be construed as limiting the scope of the present technology, as defined by the appended claims. The examples can include or incorporate any of the variations, aspects, or embodiments of the present technology described above. The variations, aspects, or embodiments described above may also further each include or incorporate the variations of any or all other variations, aspects or embodiments of the present technology.

### Materials and Instrumentation

All media and reagents were obtained from commercial sources (Sigma-Aldrich, Teknova, Research Products International, New England Biolabs, and Thermo Fisher Scientific). Synthetic oligos were purchased from Integrated DNA Technologies.

Supernatant samples were lyophilized in a FreeZone Freeze Dryer (Labconco). Fluorescence (EX 500 nm/EM 540 nm) and OD₆₀₀ were read on a Synergy™ 2 plater reader (BioTek). Data was analyzed using Prism 9 software (GraphPad by Dotmatics).

### Example 1. Construction of Inducible Strains

P. megaterium Strains

To make plasmids for AIP-analog expression in P. megaterium, the agrBD locus was amplified from wild type (WT) S. aureus with either the group I or III type agr system (Sa-G1 and Sa-G3). The amplicon and the pT7-RNAP plasmid (MoBiTec GmbH) were digested with SpeI and BamHI, purified, and ligated together. Site-directed mutagenesis was used to introduce the D29A mutation into the AIP region of the agrD genes, producing the pT7-G1BD_D5A (Group I) and pT7-G3BD_D4A (Group III) plasmids. The plasmids were transformed into P. megaterium using established methods.²

B. subtilis Strains

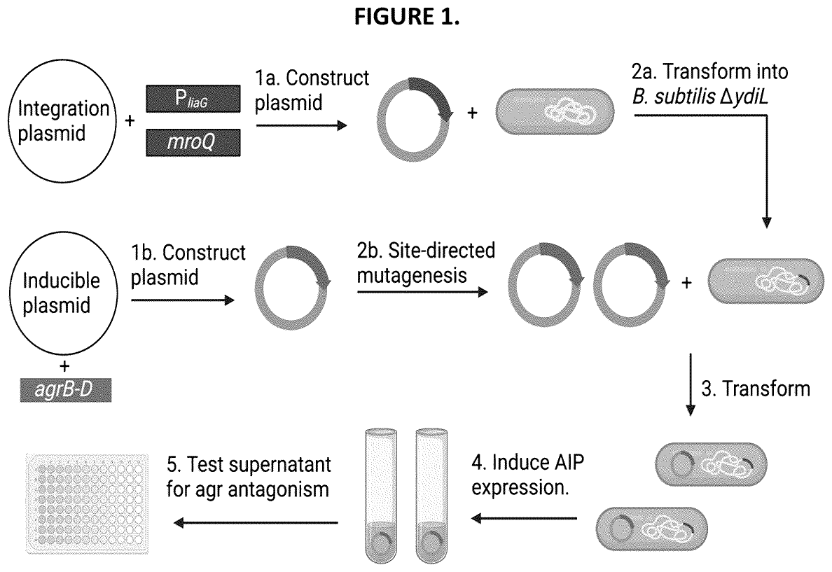

To construct B. subtilis strains that express MroQ (Bs-ΔydiL-mroQ), the P_{liaG} promoter and the S. aureus mroQ gene were cloned into the pBS1C plasmid using BioBrick cloning (FIG. 1). The plasmid was transformed into Bs-ΔydiL and transformants were selected for on LB agar with 5 μg/ml of chloramphenicol.

Plasmids for AIP and AIP-analog expression in B. subtilis were made using the Bacillus BioBrick system (FIG. 1).^{3,4} The agrBD loci were amplified from WT S. aureus and inserted into the pBS0EXylRPxylA_{(V2)} plasmid. The BioBrick system uses a PstI restriction site. To avoid issues with a PstI restriction site natively found in agrB, a restriction site for NsiI, which generates compatible cohesive ends to PstI, was added to the reverse primers DLW108 and DLW111 for agrBD amplification. Site-directed mutagenesis was then used to introduce the D29A mutation to the agrD gene. Plasmids for native and non-native AIP production were transformed into Bs-ΔydiL-mroQ using established methods.⁵

### Example 2. Construction of Constitutive Strains

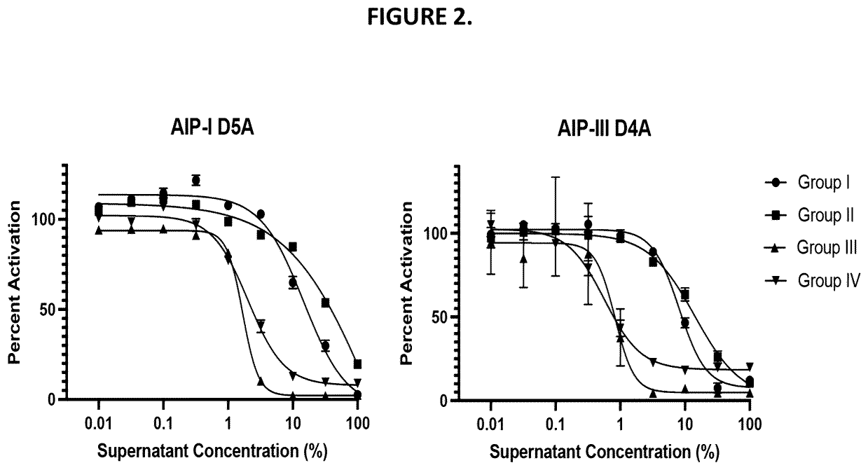

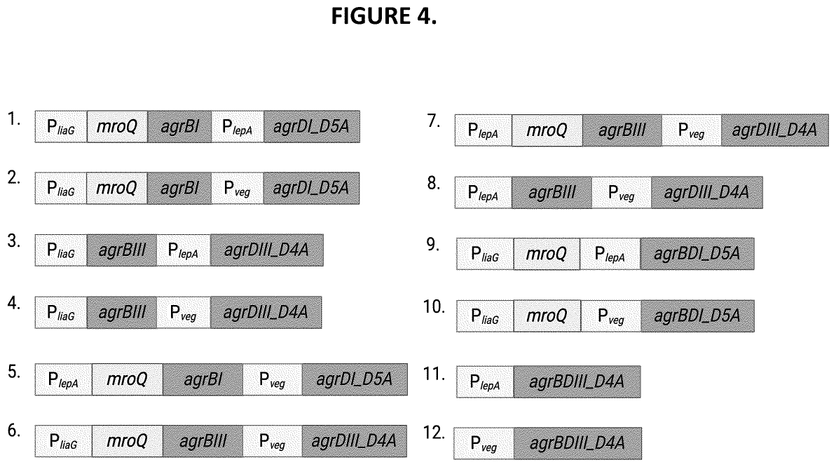

Strains of B. subtilis that constitutively express either AIP-I D5A or AIP-III D4A were engineered using the Bacillus BioBrick system.^{3,4} Genes for P_{liaG}, mroQ, agrB, P_{lepA}/P_{veg}, and agrD were cloned into the pBS1C plasmid in the arrangements shown in FIG. 2. The B. subtilis ribosome-binding sequence AAGGAGG and a 7-nucleotide spacer were added 5′ of each protein coding gene. A terminator stem was added to the end of the agrD gene to prevent polar effects. The agrB genes in groups I and III S. aureus naturally contains a PstI restriction site. To keep this site from interfering with restriction cloning, site-directed mutagenesis was performed to alter the sequence without introducing amino acid changes. The genetic modules in FIG. 4 were integrated into the genome of B. subtilis.

### Example 3. Production of AIP-Containing Supernatant

In both P. megaterium and B. subtilis strains produced in Example 1, AIP production was induced by diluting overnight cultures 1:100 in LB containing 1% xylose. After 8 hours the cultures were centrifuged, and the supernatant was purified with a 0.22 μm filter. The supernatant was then lyophilized and reconstituted in sterile milli-Q water at 10× the original concentration.

B. subtilis strains produced in Example 2 were grown overnight, diluted 1:100 in fresh media, and grown for 6 hours before supernatant was collected.

### Example 4: Staphylococcus aureus Reporter Assays

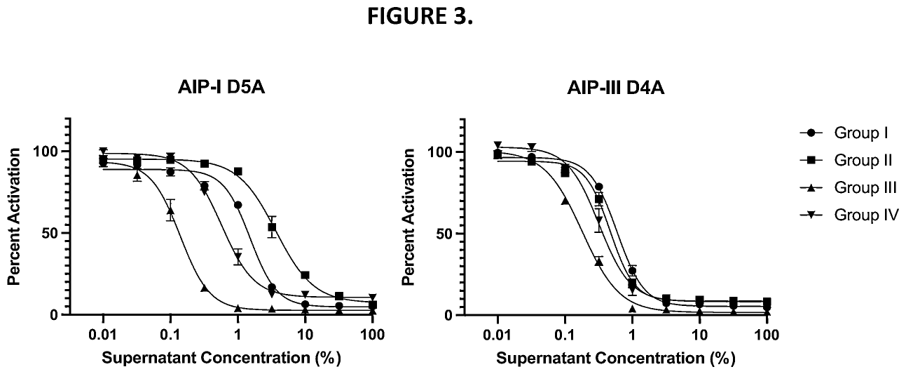

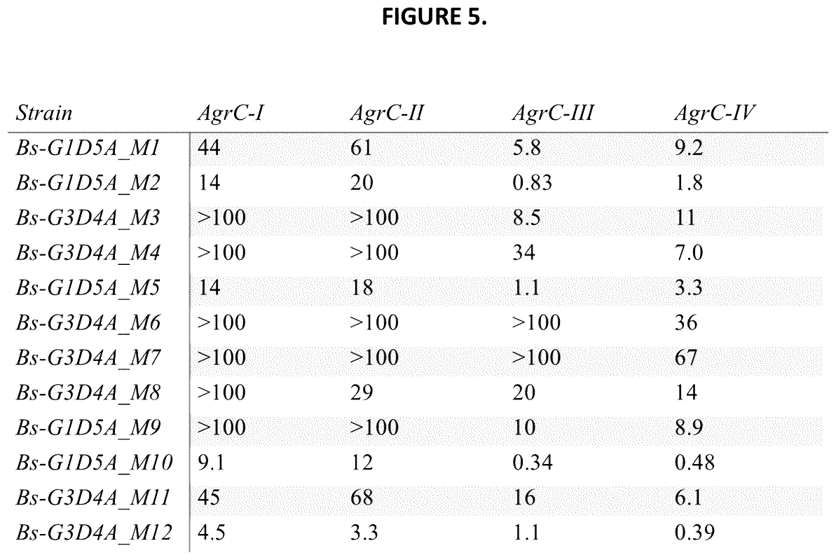

Groups I-IV S. aureus strains containing the pDB59 plasmid were used to conduct agr antagonism assays. Assays were conducted as previously described,¹ with the following adjustments: 10×WT P. megaterium or B. subtilis supernatant was used as a vehicle control instead of DMSO and 20 μl of sample/vehicle control and 180 μl of 1:50 overnight S. aureus in brain heart infusion (BHI) growth medium was added to each well for inducible strains P. megaterium (FIG. 2) and B. subtilis (FIG. 3) from Example 1 and constitutive strain B. subtilis (FIG. 5) from Example 2.

### Example 5: Competition Assays

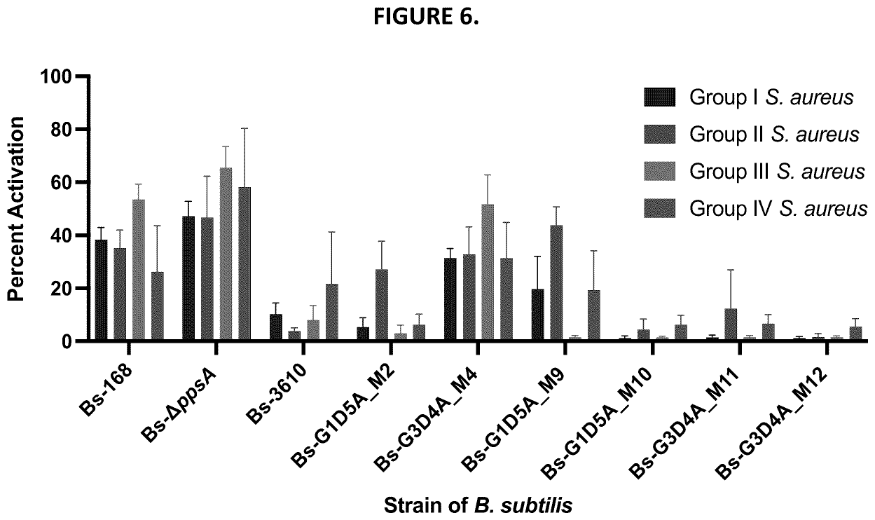

Competition assays were performed to test agr antagonism in co-cultures of B. subtilisstrains produced in Example 2 and S. aureus. Overnight cultures of the B. subtilis were grown for 16 hours in LB, and S. aureus were grown for 20 hours in BHI. Each overnight culture was then diluted 1:200 in fresh BHI. 100 μl each of fresh B. subtilis and S. aureus were added to 96 well plates and incubated shaking at 37° C. After 24 hours, fluorescence (EX 500 nm/EM 540 nm) and OD₆₀₀ were read using a plate reader (FIG. 6).

TABLE 1
Primers used in cloning and strain preparation.

| Primer Name | Description | Sequence |
|---|---|---|
| DLW001 | Group I agrBD for Pm*, forward | ACTGACACTAGTATGAATTATTTTGATAATA |
| | | AAATTGACCAG (SEQ ID NO: 1) |
| DLW052 | Group I agrBD for Pm, reverse | CGAATTGGATCCCACCTACTATCACACTC |
| | | (SEQ ID NO: 2) |
| DLW005 | Group III agrBD for Pm, forward | ACTGACACTAGTATGAATTATTTTGATAACA |
| | | AAATTGACCAG (SEQ ID NO: 3) |
| DLW054 | Group III agrBD for Pm, reverse | CGAATTGGATCCCACACTTTCTCTTATAGTTA |
| | | TTCGTG (SEQ ID NO: 4) |
| DLW007 | AIP-I D5A, mutagenesis | CAACTTCATCCATTATGAAGGCACAAGTACT |
| | | ATAAGCTGCG (SEQ ID NO: 5) |
| DLW008 | AIP-I D5A, mutagenesis | CGCAGCTTATAGTACTTGTGCCTTCATAATGG |
| | | ATGAAGTTG (SEQ ID NO: 6) |
| DLW011 | AIP-III D4A, mutagenesis | CTTCAGCTTCATCCAATAAAAAAGCACAATT |
| | | TATATAGGCTGCTCTG (SEQ ID NO: 7) |
| DLW012 | AIP-III D4A, mutagenesis | CAGAGCAGCCTATATAAATTGTGCTTTTTTAT |
| | | TGGATGAAGCTGAAG (SEQ ID NO: 8) |
| DLW121 | Group I agrBD for BioBrick, | CGAATTTCTAGAAAGGAGGCTGACTAATGAA |
| | forward | TTATTTTGATAATAAAATTGACCAG (SEQ ID |
| | | NO: 9) |
| DLW108 | Group I agrBD for BioBrick, | AGCAGATGCATCACCTACTATCACACTC |
| | reverse | (SEQ ID NO: 10) |
| DLW122 | Group III agrBD for BioBrick, | CGAATTTCTAGAAAGGAGGCTGACTAATGAA |
| | forward | TTATTTTGATAACAAAATTGACCAG (SEQ ID |
| | | NO: 11) |
| DLW111 | Group III agrBD for BioBrick, | AGCAGATGCATCACACTTTCTCTTATAGTTAT |
| | reverse | TCGTG (SEQ ID NO: 12) |
| DLW120 | mroQ for BioBrick, forward | CGAATTTCTAGAAAGGAGGCTGACTAATGAC |
| | | AAGATTATGGGCATCATTGC (SEQ ID NO: 13) |
| DLW113 | mroQ with artificial terminator | GGACTCTGCAGCGGCCGCTACTAGTAGCTTT |
| | stem for BioBrick, reverse | TTTTCGCACCCTAATGGGTGCGTATCTTATGG |
| | | AATAAAAATGTGATATATAAAATTCGC (SEQ |
| | | ID NO: 14) |
| DLW127 | mroQ for BioBrick, reverse | GGACTCTGCAGCGGCCGCTACTAGTAGCTTA |
| | | TGGAATAAAAATGTGATATATAAAATTCGC |
| | | (SEQ ID NO: 15) |
| DLW123 | Group I agrB, for editing out PstI | GTGTAATCTCAGTATATGCTCCTGCTGCAACT |
| | | AAAAAGAAGCCCATTCC (SEQ ID NO: 16) |
| DLW124 | Group I agrB, for editing out PstI | GGAATGGGCTTCTTTTTAGTTGCAGCAGGAG |
| | | CATATACTGAGATTACAC (SEQ ID NO: 17) |
| DLW125 | Group III agrB, for editing out | GTGTTGGCGTAGTGATTAAATATGCACCTGC |
| | PstI | TGCTACTAAGAAGAAGCCTATCC (SEQ ID |
| | | NO: 18) |
| DLW126 | Group III agrB, for editing out | GGATAGGCTTCTTCTTAGTAGCAGCAGGTGC |
| | PstI | ATATTTAATCACTACGCCAACAC (SEQ ID |
| | | NO: 19) |
| DLW128 | Group I agrB for BioBrick, | GATCCGAATTCGCGGCCGCTTCTAGAGAAGG |
| | forward | AGGCTGACTAATGAATTATTTTGATAATAAA |
| | | ATTGACCAG (SEQ ID NO: 20) |
| DLW129 | Group I agrB for BioBrick, | GGACTCTGCAGCGGCCGCTACTAGTATCATT |
| | reverse | TTAAGTCCTCCTTAATAAAG (SEQ ID NO: 21) |
| DLW130 | Group III agrB for BioBrick, | GATCCGAATTCGCGGCCGCTTCTAGAGAAGG |
| | forward | AGGCTGACTAATGAATTATTTTGATAACAAA |
| | | ATTGACCAG (SEQ ID NO: 22) |
| DLW131 | Group III agrB for BioBrick, | GGACTCTGCAGCGGCCGCTACTAGTATAGTC |
| | reverse | CTCCTTTGAATAGTATATTGG (SEQ ID NO: 23) |
| DLW132 | Group I agrD for BioBrick, | GATCCGAATTCGCGGCCGCTTCTAGAGAAGG |
| | forward | AGGCTGACTAATGAATACATTATTTAACTTA |
| | | TTTTTTG (SEQ ID NO: 24) |
| DLW133 | Group I agrD with artificial | GGACTCTGCAGCGGCCGCTACTAGTATTTTTT |
| | terminator stem for BioBrick, | TCGCACCCTAATGGGTGCGTATCCACCTACT |
| | reverse | ATCACACTC (SEQ ID NO: 25) |
| DLW134 | Group III agrD for BioBrick, | GATCCGAATTCGCGGCCGCTTCTAGAGAAGG |
| | forward | AGGCTGACTAATGAAAAAATTACTCAACAAA |
| | | GTTATTGAG (SEQ ID NO: 26) |
| DLW135 | Group III agrD with artificial | GGACTCTGCAGCGGCCGCTACTAGTATTTTTT |
| | terminator stem for BioBrick, | TCGCACCCTAATGGGTGCGTATCCACACTTTC |
| | reverse | TCTTATAGTTATTCGTG (SEQ ID NO: 27) |

*Pm = Priestia megaterium

TABLE 2
Plasmids used in this study.

| | | Reference |
|---|---|---|
| Plasmid | Description | or source |
| pT7-RNAP | Plasmid for xylose-inducible gene expression in | MoBiTec |
| | Priestia megaterium. | GmbH |
| pT7-G1BD_D5A | Plasmid for xylose-inducible expression of AIP-I | This study |
| | D5A in P. megaterium. | |
| pT7-G3BD_D4A | Plasmid for xylose-inducible expression of AIP- | This study |
| | III D4A in P. megaterium. | |
| pBS0EXylRPxylA_{(V2)} | Plasmid for xylose-inducible gene expression in | BGSC*; ²⁷ |
| | B. subtilis. | |
| pBS1C | Integration plasmid for B. subtilis. | BGSC;²⁸ |
| pLiaG | Plasmid with the P_{liaG} promotor and BioBrick | BGSC;²⁸ |
| | cloning sites. | |
| pVeg | Plasmid with the P_{veg} promotor and BioBrick | BGSC;²⁸ |
| | cloning sites. | |
| pLepA | Plasmid with the P_{lepA} promotor and BioBrick | BGSC;²⁸ |
| | cloning sites. | |
| pBS1C-pliaG-MroQ | Plasmid for integrating P_{liaG}-mroQ into the B. | This study |
| | subtilis genome. | |
| pBS0EXylRPxylA_{(V2)}- | Plasmid for xylose-inducible expression of AIP-I | This study |
| G1BD | in B. subtilis. | |
| pBS0EXylRPxylA_{(V2)}- | Plasmid for xylose-inducible expression of AIP- | This study |
| G3BD | III in B. subtilis. | |
| pBS0EXylRPxylA_{(V2)}- | Plasmid for xylose-inducible expression of AIP- | This study |
| G1BD_D5A | I D5A in B. subtilis. | |
| pBS0EXylRPxylA_{(V2)}- | Plasmid for xylose-inducible expression of AIP- | This study |
| G3BD_D4A | III D4A in B. subtilis. | |
| pBS1C-PliaG-MroQ- | Plasmid for integrating module 1 (FIG. 4) into | This study |
| AgrBI-PlepA- | the B. subtilis genome. | |
| AgrD1_D5A | | |
| pBS1C-PliaG-MroQ- | Plasmid for integrating module 2 (FIG. 4) into | This study |
| AgrBI-Pveg-AgrD1_D5A | the B. subtilis genome. | |
| pBS1C-PliaG-AgrBIII- | Plasmid for integrating module 3 (FIG. 4) into | This study |
| PlepA-AgrD3_D4A | the B. subtilis genome. | |
| pBS1C-PliaG-AgrBIII- | Plasmid for integrating module 4 (FIG. 4) into | This study |
| Pveg-AgrD3_D4A | the B. subtilis genome. | |
| pBS1C-PlepA-MroQ- | Plasmid for integrating module 5 (FIG. 4) into | This study |
| AgrBI-Pveg-AgrDI_D5A | the B. subtilis genome. | |
| pBS1C-PliaG-MroQ- | Plasmid for integrating module 6 (FIG. 4) into | This study |
| AgrBIII-Pveg- | the B. subtilis genome. | |
| AgrDIII_D4A | | |
| pBS1C-PlepA-MroQ- | Plasmid for integrating module 7 (FIG. 4) into | This study |
| AgrBIII-Pveg- | the B. subtilis genome. | |
| AgrDIII_D4A | | |
| pBS1C-PlepA-AgrBIII- | Plasmid for integrating module 8 (FIG. 4) into | This study |
| Pveg-AgrDIII_D4A | the B. subtilis genome. | |
| pBS1C-PliaG-MroQ- | Plasmid for integrating module 9 (FIG. 4) into | This study |
| PlepA-AgrBDI_D5A | the B. subtilis genome. | |
| pBS1C-PliaG-MroQ- | Plasmid for integrating module 10 (FIG. 4) into | This study |
| Pveg-AgrBDI_D5A | the B. subtilis genome. | |
| pBS1C-PlepA- | Plasmid for integrating module 11 (FIG. 4) into | This study |
| AgrBDIII_D4A | the B. subtilis genome. | |
| pBS1C-Pveg- | Plasmid for integrating module 12 (FIG. 4) into | This study |
| AgrBDIII_D4A | the B. subtilis genome. | |

*BGSC = Bacillus genetic stock center

TABLE 3
Bacterial strains used in this study.

| | | | Plasmid/Genetic | Reference |
|---|---|---|---|---|
| Strain Name | Species | Strain | Insertion or Deletion | or source |
| Pm-MS941 | Priestia | MS941 | None | MoBiTec |
| | megaterium | | | GmbH |
| Pm-G1D5A | Priestia | MS941 | pT7-G1BD_D5A | This study |
| | megaterium | | | |
| Pm-G3D4A | Priestia | MS941 | pT7-G3BD_D4A | This study |
| | megaterium | | | |
| Bs-168 | Bacillus | 168 | None | BGSC* |
| | subtilis | | | |
| Bs-ΔydiL | Bacillus | 168 | ΔydiL::kan | BGSC |
| | subtilis | | | |
| Bs-ΔppsA | Bacillus | 168 | ΔppsA::kan | BGSC |
| | subtilis | | | |
| Bs-3610 | Bacillus | NCIB3610 | None | BGSC |
| | subtilis | | | |
| Bs-ΔydiL-mroQ | Bacillus | 168 | ΔydiL::kan, mroQ::cam | This study |
| | subtilis | | | |
| Bs-G1 | Bacillus | 168 | pBS0EXylRPxylA_{(V2)}- | This study |
| | subtilis | | G1BD | |
| Bs-G3 | Bacillus | 168 | pBS0EXylRPxylA_{(V2)}- | This study |
| | subtilis | | G3BD | |
| Bs-G1D5A | Bacillus | 168 | pBS0EXylRPxylA_{(V2)}- | This study |
| | subtilis | | G1BD_D5A | |
| Bs-G3D4A | Bacillus | 168 | pBS0EXylRPxylA_{(V2)}- | This study |
| | subtilis | | G3BD_D4A | |
| Bs-G1D5A_M1 | Bacillus | 168 | Module 1 (FIG. 4) | This study |
| | subtilis | | | |
| Bs-G1D5A_M2 | Bacillus | 168 | Module 2 (FIG. 4) | This study |
| | subtilis | | | |
| Bs-G3D4A_M3 | Bacillus | 168 | Module 3 (FIG. 4) | This study |
| | subtilis | | | |
| Bs-G3D4A_M4 | Bacillus | 168 | Module 4 (FIG. 4) | This study |
| | subtilis | | | |
| Bs-G1D5A_M5 | Bacillus | 168 | Module 5 (FIG. 4) | This study |
| | subtilis | | | |
| Bs-G3D4A_M6 | Bacillus | 168 | Module 6 (FIG. 4) | This study |
| | subtilis | | | |
| Bs-G3D4A_M7 | Bacillus | 168 | Module 7 (FIG. 4) | This study |
| | subtilis | | | |
| Bs-G3D4A_M8 | Bacillus | 168 | Module 8 (FIG. 4) | This study |
| | subtilis | | | |
| Bs-G1D5A_M9 | Bacillus | 168 | Module 9 (FIG. 4) | This study |
| | subtilis | | | |
| Bs-G1D5A_M10 | Bacillus | 168 | Module 10 (FIG. 4) | This study |
| | subtilis | | | |
| Bs-G3D4A_M11 | Bacillus | 168 | Module 11 (FIG. 4) | This study |
| | subtilis | | | |
| Bs-G3D4A_M12 | Bacillus | 168 | Module 12 (FIG. 4) | This study |
| | subtilis | | | |
| Sa-G1YFP | Staphylococcus | AH1677 | pDB59 | ^{6, 7} |
| | aureus | | | |
| Sa-G2YFP | Staphylococcus | AH430 | pDB59 | ⁶⁻⁸ |
| | aureus | | | |
| Sa-G3YFP | Staphylococcus | AH1747 | pDB59 | ^{6, 7} |
| | aureus | | | |
| Sa-G4YFP | Staphylococcus | AH1872 | pDB59 | ^{6, 7} |
| | aureus | | | |
| Sa-G1 | Staphylococcus | RN6390 | None | ⁹ |
| | aureus | | | |
| Sa-G3 | Staphylococcus | RN8463 | None | ¹⁰ |
| | aureus | | | |

*BGSC = Bacillus genetic stock center

### Partial Sequences List

T7-RNAP plasmid
SEQ ID NO: 28
TGTCACGGAGGTTCAAGTTACCTTTAGCCGGAAGTGCTGGCATTTTGTCCAATTG
AGACTCGTGCAACTGGTCAGCGAACTGGTCGTAGAAATCAGCCAGTACATCACA
AGACTCATATGTGTCAACCATAGTTTCGCGCACTGCTTTGAACAGGTTCGCAGCG
TCAGCCGGAATGGTACCGAAGGAGTCGTGAATCAGTGCAAAAGATTCGATTCCG
TACTTCTCGTGTGCCCACACTACAGTCTTACGAAGGTGGCTACCGTCTTGGCTGT
GTACAAAGTTAGGAGCGATACCAGACTCCTGTTTGTGTGCATCAATCTCGCTATC
TTTGTTGGTGTTAATGGTAGGCTGTAAGCGGAACTGACCGAGGAACATCAGGTTC
AAGCGCGTCTGAATAGGCTTCTTGTATTCCTGCCACACAGGGAAACCATCAGGAG
TTACCCAATGCACAGCGCAACGCTTGCGAAGAATCTCTCCAGTCTTCTTATCTTTG
ACCTCAGCAGCCAGCAGCTTAGCAGCAGACTTAAGCCAGTTCATTGCTTCAACCG
CAGCTACCACCGTCACGCTCACAGATTCCCAAATCAGCTTAGCCATGTATCCAGC
AGCCTGATTCGGCTGAGTGAACATCAGACCCTTGCCGGAATCAATAGCTGGCTGA
ATGGTATCTTCCAGCACTTGTTGACGGAAGCCGAACTCTTTGGACCCGTAAGCCA
GCGTCATGACTGAACGCTTAGTCACACTGCGAGTAACACCGTAAGCCAGCCATTG
ACCAGCCAGTGCCTTAGTGCCCAGCTTGACTTTCTCAGAGATTTCACCAGTGTTCT
CATCGGTCACGGTAACTACTTCGTTATCGGTCCCATTGATTGCGTCTGCTTGTAGA
ATCTCGTTGACTTTCTTAGCAACAATCCCGTAGATGTCCTGAACGGTTTCACTAG
GAAGCAAGTTAACCGCGCGACCACCTACCTCATCTCGGAGCATCGCGGAGAAGT
GCTGGATGCCAGAGCAAGACCCGTCAAACGCCAGCGGAAGGGAGCAGTTATAGC
TCAGGCCGTGGTGCTGTACCCCAGCGTACTCAAAGCAGAACGCAAGGAAGCAGA
ACGGAGAATCTTGCTCAGCCCACCAAGTGTTCTCCAGTGGAGACTTAGCGCAAGC
CATGATGTTCTCGTGGTTTTCCTCAATGAACTTGATGCGCTCAGGGAACGGAACC
TTATCGACACCCGCACAGTTTGCACCGTGGATTTTCAGCCAGTAGTAACCTTCCTT
ACCGATTGGTTTACCTTTCGCCAGCGTAAGCAGTCCTTTGGTCATATCGTTACCTT
GCGGGTTGAACATTGACACAGCGTAAACACGACCGCGCCAGTCCATGTTGTAAG
GGAACCAGATGGCCTTATGGTTAGCAAACTTATTGGCTTGCTCAAGCATGAACTC
AAGGCTGATACGGCGAGACTTGCGAGCCTTGTCCTTGCGGTACACAGCAGCGGC
AGCACGTTTCCACGCGGTGAGAGCCTCAGGATTCATGTCGATGTCTTCCGGTTTC
ATCGGGAGTTCTTCACGCTCAATCGCAGGGATGTCCTCGACCGGACAATGCTTCC
ACTTGGTGATTACGTTGGCGACCGCTAGGACTTTCTTGTTGATTTTCCATGCGGTG
TTTTGCGCAATGTTAATCGCTTTGTACACCTCAGGCATGTAAACGTCTTCGTAGCG
CATCAGTGCTTTCTTACTGTGAGTACGCACCAGCGCCAGAGGACGACGACCGTTA
GCCCAATAGCCACCACCAGTAATGCCAGTCCACGGCTTAGGAGGAACTACGCAA
GGTTGGAACATCGGAGAGATGCCAGCCAGCGCACCTGCACGGGTTGCGATAGCC
TCAGCGTATTCAGGTGCGAGTTCGATAGTCTCAGAGTCTTGACCTACTACGCCAG
CATTTTGGCGGTGTAAGCTAACCATTCCGGTTGACTCAATGAGTCATCTCGATGC
AGCGTACTCCTACATGAATAGAGTCTTCCTTATGCCACGAAGACCACGCCTCGCC
ACCGAGTAGACCCTTAGAGAGCATGTCAGCCTCGACAACTTGCATAAATGCTTTC
TTGTAGACGTGCCCTACGCGCTTGTTGAGTTGTTCCTCAACGTTTTTCTTGAAGTG
CTTAGCTTCAAGGTCACGGATACGACCGAAGCGAGCCTCGTCCTCAATGGCCCGA
CCGATTGCGCTTGCTACAGCCTGAACGGTTGTATTGTCAGCACTGGTTAGGCAAG
CCAGAGTGGTCTTAATGGTGATGTACGCTACGGCTTCCGGCTTGATTTCTTGCAG
GAACTGGAAGGCTGTCGGGTGCTTGCCGCGCTTAGCTTTCACTTCCTCAAACCAG
TCGTTGATGCGTGCAATCATCTTAGGGAGTAGGGTAGTGATGAGAGGCTTGGCG
GCAGCGTTATCCGCAACCTCACCAGCTTTAAGTTGACGCTCAAACATCTTGCGGA
AGCGTGCTTCACCCATCTCGTAAGACTCATGCTCAAGGGCCAACTGTTCGCGAGC
TAAACGCTCACCGTAATGGTCAGCCAGAGTGTTGAACGGGATAGCAGCCAGTTC
GATGTCAGAGAAGTCGTTCTTAGCGATGTTAATCGTGTTCATTCCTCCTCCTCCAC
TAGTTTGGACCATTTGTCATTTCCCCCTTTGATTTAAGTGAACAAGTTTATCCATC
AACTATCTTAATTGAGTTAGTTTGTTTATCCAATAAACTAACTTTATCTCATCATA
TACAAAATAAATGTTTATTTCAATGTTTTTTTTAGAAAATTTAGTTATAATATTAG
ATATGATACTTTTAAATATCTAATTCAAGCTTCAAAAAACACCAACTTAGTTCGG
TGGATAAACAAAGGAGTGGTTATTATTCAAATTGCAGATCAAGCTTTAGTAAAA
AAAATGAATCAAAAATTAATATTAGATGAAATTTTGAAGAACTCCCCTGTCTCCA
GGGCAACTCTCTCTGAGATTACAGGATTAAACAAGTCTACTGTCTCCTCTCAAGT
AAATACACTGCTTGAAAAAGATTTTATTTTTGAAATTGGGGCAGGGCAATCTAGA
GGCGGCAGAAGACCTGTAATGCTTGTTTTTAATAAGAATGCAGGCTACTCGATTG
GTATTGATATAGGAGTCGACTATCTTAACGGAATTCTAACCGACTTAGAAGGAAA
TATTATTCTCGAGAAGACTTCTGACTTGTCTAGTTCTTCCGCTAGTGAAGTAAAA
GAGATTTTATTTGCACTTATTCATGGTTTTGTAACCCATATGCCTGAGTCCCCTTA
TGGTCTAGTCGGAATAGGAATTTGTGTTCCAGGCCTTGTAGATCGTCATCAGCAA
ATTATTTTCATGCCTAACTTAAATTGGAATATCAAAGATTTGCAGTTTTTAATTGA
GAGTGAGTTTAATGTTCCGGTTTTTGTTGAAAATGAAGCTAATGCAGGAGCATAC
GGTGAAAAAGTATTTGGTATGACAAAAAACTATGAAAACATCGTTTACATCAGT
ATTAATATCGGAATTGGAACTGGACTTGTTATTAACAACGAATTGTATAAAGGTG
TTCAGGGTTTTTCTGGGGAAATGGGTCATATGACGATAGATTTTAATGGACCCAA
ATGCAGCTGTGGAAATCGAGGCTGTTGGGAATTATATGCTTCTGAAAAAGCGTTA
CTGGCTTCGCTCTCTAAAGAAGAAAAGAATATTTCTCGAAAAGAGATTGTGGAA
CGCGCAAATAAAAATGATGTAGAAATGTTAAATGCACTTCAAAACTTTGGCTTTT
ATATCGGAATTGGATTAACCAATATCCTTAATACATTTGATATAGAAGCTGTTAT
CTTGAGAAATCATATAATTGAATCTCATCCCATTGTTTTAAATACGATTAAAAAC
GAAGTTTCTTCTAGAGTCCATTCTCATTTAGACAATAAATGTGAACTATTGCCTTC
TTCGTTAGGAAAAAATGCACCTGCTTTAGGAGCGGTTTCTATCGTTATTGATTCTT
TTTTAAGTGTTACCCCTATAAGTTAGTTAAGTGAACGCAAAGGGCGCCTGATGCG
GTATTTTCTCCTTACGCATCTGTGCGGTATTTCACACCGCATAGATCATTATTTAG
CGATGTCGCCTACTCCTGAGGACGATCCGGCAATAGTTACCCTTATTATCAAGAT
AAGAAAGAAAAGGATTTTTCGCTACGCTCAAATCCTTTAAAAAAACACAAAAGA
CCACATTTTTTAATGTGGTCTTTTATTCTTCAACTAAAGCACCCATTAGTTCAACA
AACGAAAATTGGATAAAGTGGGATATTTTTAAAATATATATTTATGTTACAGTAA
TATTGACTTTTAAAAAAGGATTGATTCTAATGAAGAAAGCAGACAAGTAAGCCT
CCTAAATTCACTTTAGATAAAAATTTAGGAGGCATATCAAATGAACTTTAATAAA
ATTGATTTAGACAATTGGAAGAGAAAAGAGATATTTAATCATTATTTGAACCAAC
AAACGACTTTTAGTATAACCACAGAAATTGATATTAGTGTTTTATACCGAAACAT
AAAACAAGAAGGATATAAATTTTACCCTGCATTTATTTTCTTAGTGACAAGGGTG
ATAAACTCAAATACAGCTTTTAGAACTGGTTACAATAGCGACGGAGAGTTAGGTT
ATTGGGATAAGTTAGAGCCACTTTATACAATTTTTGATGGTGTATCTAAAACATT
CTCTGGTATTTGGACTCCTGTAAAGAATGACTTCAAAGAGTTTTATGATTTATACC
TTTCTGATGTAGAGAAATATAATGGTTCGGGGAAATTGTTTCCCAAAACACCTAT
ACCTGAAAATGCTTTTTCTCTTTCTATTATTCCATGGACTTCATTTACTGGGTTTA
ACTTAAATATCAATAATAATAGTAATTACCTTCTACCCATTATTACAGCAGGAAA
ATTCATTAATAAAGGTAATTCAATATATTTACCGCTATCTTTACAGGTACATCATT
CTGTTTGTGATGGTTATCATGCAGGATTGTTTATGAACTCTATTCAGGAATTGTCA
GATAGGCCTAATGACTGGCTTTTATAATATGAGATAATGCCGACTGTACTTTTTA
CAGTCGGTTTTCTAATGTCACTAACCTGCCCCGTTAGTTGAAGAAGGTTTTTATAT
TACAGCTCCAGATCTATGGTGCACTCTCAGTACAATCTGCTCTGATGCCGCATAG
TTAAGCCAGCCCCGACACCCGCCAACACCCGCTGACGCGCCCTGACGGGCTTGTC
TGCTCCCGGCATCCGCTTACAGACAAGCTGTGACCGTCTCCGGGAGCTGCATGTG
TCAGAGGTTTTCACCGTCATCACCGAAACGCGCGAGACGAAAGGGCCTCGTGAT
ACGCCTATTTTTATAGGTTAATGTCATGATAATAATGGTTTCTTAGACGTCAGGTG
GCACTTTTCGGGGAAATGTGCGCGGAACCCCTATTTGTTTATTTTTCTAAATACAT
TCAAATATGTATCCGCTCATGAGACAATAACCCTGATAAATGCTTCAATAATATT
GAAAAAGGAAGAGTATGAGTATTCAACATTTCCGTGTCGCCCTTATTCCCTTTTTT
GCGGCATTTTGCCTTCCTGTTTTTGCTCACCCAGAAACGCTGGTGAAAGTAAAAG
ATGCTGAAGATCAGTTGGGTGCACGAGTGGGTTACATCGAACTGGATCTCAACA
GCGGTAAGATCCTTGAGAGTTTTCGCCCCGAAGAACGTTTTCCAATGATGAGCAC
TTTTAAAGTTCTGCTATGTGGCGCGGTATTATCCCGTATTGACGCCGGGCAAGAG
CAACTCGGTCGCCGCATACACTATTCTCAGAATGACTTGGTTGAGTACTCACCAG
TCACAGAAAAGCATCTTACGGATGGCATGACAGTAAGAGAATTATGCAGTGCTG
CCATAACCATGAGTGATAACACTGCGGCCAACTTACTTCTGACAACGATCGGAG
GACCGAAGGAGCTAACCGCTTTTTTGCACAACATGGGGGATCATGTAACTCGCCT
TGATCGTTGGGAACCGGAGCTGAATGAAGCCATACCAAACGACGAGCGTGACAC
CACGATGCCTGTAGCAATGGCAACAACGTTGCGCAAACTATTAACTGGCGAACT
ACTTACTCTAGCTTCCCGGCAACAATTAATAGACTGGATGGAGGCGGATAAAGTT
GCAGGACCACTTCTGCGCTCGGCCCTTCCGGCTGGCTGGTTTATTGCTGATAAAT
CTGGAGCCGGTGAGCGTGGGTCTCGCGGTATCATTGCAGCACTGGGGCCAGATG
GTAAGCCCTCCCGTATCGTAGTTATCTACACGACGGGGAGTCAGGCAACTATGGA
TGAACGAAATAGACAGATCGCTGAGATAGGTGCCTCACTGATTAAGCATTGGTA
ACTGTCAGACCAAGTTTACTCATATATACTTTAGATTGATTTAAAACTTCATTTTT
AATTTAAAAGGATCTAGGTGAAGATCCTTTTTGATAATCTCATGACCAAAATCCC
TTAACGTGAGTTTTCGTTCCACTGAGCGTCAGACCCCGTAGAAAAGATCAAAGGA
TCTTCTTGAGATCCTTTTTTTCTGCGCGTAATCTGCTGCTTGCAAACAAAAAAACC
ACCGCTACCAGCGGTGGTTTGTTTGCCGGATCAAGAGCTACCAACTCTTTTTCCG
AAGGTAACTGGCTTCAGCAGAGCGCAGATACCAAATACTGTTCTTCTAGTGTAGC
CGTAGTTAGGCCACCACTTCAAGAACTCTGTAGCACCGCCTACATACCTCGCTCT
GCTAATCCTGTTACCAGTGGCTGCTGCCAGTGGCGATAAGTCGTGTCTTACCGGG
TTGGACTCAAGACGATAGTTACCGGATAAGGCGCAGCGGTCGGGCTGAACGGGG
GGTTCGTGCACACAGCCCAGCTTGGAGCGAACGACCTACACCGAACTGAGATAC
CTACAGCGTGAGCTATGAGAAAGCGCCACGCTTCCCGAAGGGAGAAAGGCGGAC
AGGTATCCGGTAAGCGGCAGGGTCGGAACAGGAGAGCGCACGAGGGAGCTTCC
AGGGGGAAACGCCTGGTATCTTTATAGTCCTGTCGGGTTTCGCCACCTCTGACTT
GAGCGTCGATTTTTGTGATGCTCGTCAGGGGGGCGGAGCCTATGGAAAAACGCC
AGCAACGCGGCCTTTTTACGGTTCCTGGCCTTTTGCTGGCCTTTTGCTCACATGTT
CTTTCCTGCGTTATCCCCTGATTCTGTGGATAACCGTATTACCGCCTTTGAGTGAG
CTGATACCGCTCGCCGCAGCCGAACGACCGAGCGCAGCGAGTCAGTGAGCGAGG
AAGCGGAAGAGCGCCCAATACGCAAACCGCCTCTCCCCGCGCGTTGGCCGATTC
ATTAATGCAGCTGGCACGACAGGTTTCCCGACTGGAAAGCGGGCAGTGAGCGCA
ACGCAATTAATGTGAGTTAGCTCACTCATTAGGCACCCCAGGCTTTACACTTTAT
GCTTCCGGCTCGTATGTTGTGTGGAATTGTGAGCGGATAACAATTTCACACAGGA
AACAGCTATGACCATGATTACGCCAAGCTTGCATGCCTGCAGGTCATTTATTAAC
GATTACCTGCTTTTAATTCTGCATCTAATTGCATAAGCTCCGCCTTCATTTTTTCCT
TTTCAGCTTCAAAATCATTATTCACTGGAAGATCTTCTGCTACTGCATTTTCATCA
TTTGTTTGTAGCCACTTTGGAACCATTTCTTTACGTGCATTACGTACTACACGTTT
CCTTTTTGTTTGCTTCGGTTTCCAATGTTTAAGACGGTATTCTAATAGTGATTTAT
ATTGCTTATTCGTATCAACGATTACAGTATCGTTAAGCATTTCACTTTTCACTTCG
TCGAAAGGTACATTATTATATTTTTCTCGTAATTTATCATCAATAAAATCACGT
GGACTACTACTAATTTTTTCATTTGTTAATTTTTCATTTGTTTCTTTTTTATTTATAGGTGT
ACGTTCTGTGTTGTACACACTGTGTTGTACAGAACGTACATCAGAAGAAATTT
GCTTATTTTGTATCTGATGTACATTTTGTACATCAGATATTTTTTCAGTGATTTCA
AGTGGCGAATGTTGTATCACGCTTAACGTCATGACGTTTTTACCTTGCGATTGTA
AGAGATTTATTTGTTCAATGACAAGCAATTCGAAATTAGCTTGCGAAATCGGAAT
ATCTGATACGGCGTAAAAATAATCTTTTTTTCCGTCAACATATGCGCTAAAACCG
ACAGCGTATTGTTTAATAGCTAATTCCTTCCAAGCAGCGTCTACATTTCTACGAG
AAAATTTGTTTTGTAACTGGGTCTTCCTAATCGTCCAACCTTCACCTAAGCTCATA
ATATGACTTAATAGACCGATAGACCGCAAGTCCTCCAGGTCCGTTTGTAATGGCG
CGTTATGTATTTGAGCATATTGCGTTGTTTTTATTGTTTTGATAATTCCATTCATTT
CTAAATTCCTCCAACGATTGATTTTTTGTTGGAGTACTTGCTATAATATACGTAAC
CAAATAAACGTACTATTGCAACAAGTACAACCAAGAGAACGGAGGCCTCAATCT
CCGTTCTTTTTTATTATATCATGTCCTATTGATGAAAATAGGAGTGGGTATAAAAT
CGGAATTTTACTAGAGACATTGATTAATTTGCAGACAGTTTTAGTTAACAGTTGA
ATATAAATGACTCTAGAGGATCCCGGGTTACGCGAACGCGAAGTCCGACTCTAA
GA
pBS1C plasmid
SEQ ID NO: 29
TGCAGTCCGGCAAAAAAGGGCAAGGTGTCAATTCTCATGTTTGACAGCTTATCAT
CGGCAATAGTTACCCTTATTATCAAGATAAGAAAGAAAAGGATTTTTCGCTACGC
TCAAATCCTTTAAAAAAACACAAAAGACCACATTTTTTAATGTGGTCTTTATTCTT
CAACTAAAGCACCCATTAGTTCAACAAACGAAAATTGGATAAAGTGGGATATTT
TTAAAATATATATTTATGTTACAGTAATATTGACTTTTAAAAAAGGATTGATTCTA
ATGAAGAAAGCAGACAAGTAAGCCTCCTAAATTCACTTTAGATAAAAATTTAGG
AGGCATATCAAATGAACTTTAATAAAATTGATTTAGACAATTGGAAGAGAAAAG
AGATATTTAATCATTATTTGAACCAACAAACGACTTTTAGTATAACCACAGAAAT
TGATATTAGTGTTTTATACCGAAACATAAAACAAGAAGGATATAAATTTTACCCT
GCATTTATTTTCTTAGTGACAAGGGTGATAAACTCAAATACAGCTTTTAGAACTG
GTTACAATAGCGACGGAGAGTTAGGTTATTGGGATAAGTTAGAGCCACTTTATAC
AATTTTTGATGGTGTATCTAAAACATTCTCTGGTATTTGGACTCCTGTAAAGAATG
ACTTCAAAGAGTTTTATGATTTATACCTTTCTGATGTAGAGAAATATAATGGTTC
GGGGAAATTGTTTCCCAAAACACCTATACCTGAAAATGCTTTTTCTCTTTCTATTA
TTCCATGGACTTCATTTACTGGGTTTAACTTAAATATCAATAATAATAGTAATTAC
CTTCTACCCATTATTACAGCAGGAAAATTCATTAATAAAGGTAATTCAATATATT
TACCGCTATCTTTACAGGTACATCATTCTGTTTGTGATGGTTATCATGCAGGATTG
TTTATGAACTCTATTCAGGAATTGTCAGATAGGCCTAATGACTGGCTTTTATAAT
ATGAGATAATGCCGACTGTACTTTTTACAGTCGGTTTTCTAATGTCACTAACCTGC
CCCGTTAGTTGAAGAAGGTTTTTATATTACAGCTCCAGATCCTCTACGCCGGACG
CATCGTGGCCGGCATCACCGGCGCCACAGGTGCGGTTGCTGGCGCCTATATCGCC
GACATCACCGATGGGGAAGATCGGGCTCGCCACTTCGGGCTCATGAGCGCTTGTT
TCGGCGTGGGTATGGTGGCAGGCCCCGTGGCCGGGGGACTGTTGGGCGCCATCT
CCTTGCATGCACCATTCCTTGCGGCGGCGGTGCTCAACGGCCTCAACCTACTACT
GGGCTGCTTCCTAATGCAGGAGTCGCATAAGGGAGAGCGTCGACATGGATGAGC
GATGATGATATCCGTTTAGGCTGGGCGGTGATAGCTTCTCGTTCAGGCAGTACGC
CTCTTTTCTTTTCCAGACCTGAGGGAGGCGGAAATGGTGTGAGGTTCCCGGGGAA
AAGCCAAATAGGCGATCGCGGGAGTGCTTTATTTGAAGATCAGGCTATCACTGC
GGTCAATAGATTTCACAATGTGATGGCTGGACAGCCTGAGGAACTCTCGAACCC
GAATGGAAACAACCAGATATTTATGAATCAGCGCGGCTCACATGGCGTTGTGCT
GGCAAATGCAGGTTCATCCTCTGTCTCTATCAATACGGCAACAAAATTGCCTGAT
GGCAGGTATGACAATAAAGCTGGAGCGGGTTCATTTCAAGTGAACGATGGTAAA
CTGACAGGCACGATCAATGCCAGGTCTGTAGCTGTGCTTTATCCTGATGATATTG
CAAAAGCGCCTCATGTTTTCCTTGAGAATTACAAAACAGGTGTAACACATTCTTT
CAATGATCAACTGACGATTACCTTGCGTGCAGATGCGAATACAACAAAAGCCGT
TTATCAAATCAATAATGGACCAGAGACGGCGTTTAAGGATGGAGATCAATTCAC
AATCGGAAAAGGAGATCCATTTGGCAAAACATACACCATCATGTTAAAAGGAAC
GAACAGTGATGGTGTAACGAGGACCGAGAAATACAGTTTTGTTAAAAGAGATCC
AGCGTCGGCCAAAACCATCGGCTATCAAAATCCGAATCATTGGAGCCAGGTAAA
TGCTTATATCTATAAACATGATGGGAGCCGAGTAATTGAATTGACCGGATCTTGG
CCTGGAAAACCAATGACTAAAAATGCAGACGGAATTTACACGCTGACGCTGCCT
GCGGACACGGATACAACCAACGCAAAAGTGATTTTTAATAATGGCAGCGCCCAA
GTGCCCGGTCAGAATCAGCCTGGCTTTGATTACGTGCTAAATGGTTTATATAATG
ACTCGGGCTTAAGCGGTTCTCTTCCCCATTGAGGGCAAGGCTAGACGGGACTTAC
CGAAAGAAACCATCAATGATGGTTTCTTTTTTGTTCATAAATCAGACAAAACTTT
TCTCTTGCAAAAGTTTGTGAAGTGTTGCACAATATAAATGTGAAATACTTCACAA
ACAAAAAGACATCAAAGAGAAACATACCCTGCAAGGATGCTGATATTGTCTGCA
TTTGCGCCGGAGCAAACCAAAAACCTGGTGAGACACGCCTTGAATTAGTAGAAA
AGAACTTGAAGATTTTCAAAGGCATCGTTAGTGAAGTCATGGCGAGCGGATTTG
ACGGCATTTTCTTAGTCGCGACGCGAGGCTGGATGGCCTTCCCCATTATGATTCTT
CTCGCTTCCGGCGGCATCGGGATGCCCGCGTTGCAGGCCATGCTGTCCAGGCAGG
TAGATGACGACCATCAGGGACAGCTTCAAGGATCGCTCGCGGCTCTTACCAGCCT
AACTTCGATCACTGGACCGCTGATCGTCACGGCGATTTATGCCGCCTCGGCGAGC
ACATGGAACGGGTTGGCATGGATTGTAGGCGCCGCCCTATACCTTGTCTGCCTCC
CCGCGTTGCGTCGCGGTGCATGGAGCCGGGCCACCTACTGAAGTGGATTTCTTTA
AGAGCTCCTTTAACTTCCTCACCAGTAGTTGTATCGGTACCATAAGTAGAAGCAG
CAACCCAAGTAGCTTTACCAGCATCCGGTTCAACCAGCATAGTAAGAATCTTACT
GGACATCGGCAGTTCTTCGAACAGTGCGCCAACTACCAGCTCTTTCTCCAGAATG
GGCTATACCTCTTTTACCTAAAGAGCATGTAACTTTACTGGATATAGCTAGAAAA
GGCTATCGGGGAGAGTGTGATGATAAGTGGGAAGGACTATATTCAAAGGTGAAA
GCACTCGTTAAGTATATGAAAAATTCTATAGAAACTTCTCTCAATTAGGCTAATT
TTATTGCAATAACAGGTGCTTACTTTTCTGGAGTTCTTTAGCAAATTTTTTTATTA
GCTGAACTTAGTATTAGTGGCCATACTCCTCCAATCCAAAGCTATTTAGAAAGAT
TACTATATCCTCAAACAGGCGGTAACCGGCCTCTTCATCGGGAATGCGCGCGACC
TTCAGCATCGCCGGCATGTCCCCCTGGCGGACGGGAAGTATCCAGCTCGAGGTCG
GGCCGCGTTGCTGGCGTTTTTCCATAGGCTCCGCCCCCCTGACGAGCATCACAAA
AATCGACGCTCAAGTCAGAGGTGGCGAAACCCGACAGGACTATAAAGATACCAG
GCGTTTCCCCCTGGAAGCTCCCTCGTGCGCTCTCCTGTTCCGACCCTGCCGCTTAC
CGGATACCTGTCCGCCTTTCTCCCTTCGGGAAGCGTGGCGCTTTCTCATAGCTCAC
GCTGTAGGTATCTCAGTTCGGTGTAGGTCGTTCGCTCCAAGCTGGGCTGTGTGCA
CGAACCCCCCGTTCAGCCCGACCGCTGCGCCTTATCCGGTAACTATCGTCTTGAG
TCCAACCCGGTAAGACACGACTTATCGCCACTGGCAGCAGCCACTGGTAACAGG
ATTAGCAGAGCGAGGTATGTAGGCGGTGCTACAGAGTTCTTGAAGTGGTGGCCT
AACTACGGCTACACTAGAAGGACAGTATTTGGTATCTGCGCTCTGCTGAAGCCAG
TTACCTTCGGAAAAAGAGTTGATAGCTCTTGATCCGGCAAACAAACCACCGCTGG
TAGCGGTGGTTTTTTTGTTTGCAAGCAGCAGATTACGCGCAGAAAAAAAGGATCT
CAAGAAGATCCTTTGATCTTTTCTACGGGGTCTGACGCTCAGTGGAACGAAAACT
CACGTTAAGGGATTTTGGTCATGAGATTATCAAAAAGGATCTTCACCTAGATCCT
TTTAAATTAAAAATGAAGTTTTAAATCAATCTAAAGTATATATGAGTAAACTTGG
TCTGACAGTTACCAATGCTTAATCAGTGAGGCACCTATCTCAGCGATCTGTCTAT
TTCGTTCATCCATAGTTGCCTGACTCCCCGTCGTGTAGATAACTACGATACGGGA
GGGCTTACCATCTGGCCCCAGTGCTGCAATGATACCGCGAGACCCACGCTCACCG
GCTCCAGATTTATCAGCAATAAACCAGCCAGCCGGAAGGGCCGAGCGCAGAAGT
GGTCCTGCAACTTTATCCGCCTCCATCCAGTCTATTAATTGTTGCCGGGAAGCTA
GAGTAAGTAGTTCGCCAGTTAATAGTTTGCGCAACGTTGTTGCCATTGCTGCCGG
CATCGTGGTGTCACGCTCGTCGTTTGGTATGGCTTCATTCAGCTCCGGTTCCCAAC
GATCAAGGCGAGTTACATGATCCCCCATGTTGTGCAAAAAAGCGGTTAGCTCCTT
CGGTCCTCCGATCGTTGTCAGAAGTAAGTTGGCCGCAGTGTTATCACTCATGGTT
ATGGCAGCACTGCATAATTCTCTTACTGTCATGCCATCCGTAAGATGCTTTTCTGT
GACTGGTGAGTACTCAACCAAGTCATTCTGAGAATAGTGTATGCGGCGACCGAG
TTGCTCTTGCCCGGCGTCAACACGGGATAATACCGCGCCACATAGCAGAACTTTA
AAAGTGCTCATCATTGGAAAACGTTCTTCGGGGCGAAAACTCTCAAGGATCTTAC
CGCTGTTGAGATCCAGTTCGATGTAACCCACTCGTGCACCCAACTGATCTTCAGC
ATCTTTTACTTTCACCAGCGTTTCTGGGTGAGCAAAAACAGGAAGGCAAAATGCC
GCAAAAAAGGGAATAAGGGCGACACGGAAATGTTGAATACTCATACTCTTCCTT
TTTCAATATTATTGAAGCATTTATCAGGGTTATTGTCTCATGAGCGGATACATATT
TGAATGTATTTAGAAAAATAAACAAATAGGGGTTCCGCGCACATTTCCCCGAAA
AGTGCCACCTGACGTCTAAGAAACCATTATTATCATGACATTAACCTATAAAAAT
AGGCGTATCACGAGGCCCTTTCGTCTTCAAGAATTAACAAAATTCTCCAGTCTTC
ACATCGGTTTGAAAGGAGGAAGCGGAAGAATGAAGTAAGAGGGATTTTTGACTC
CGAAGTAAGTCTTCAAAAAATCAAATAAGGAGTGTCAAGAATGTTTGCAAAACG
ATTCAAAACCTCTTTACTGCCGTTATTCGCTGGATTTTTATTGCTGTTTCATTTGGT
TCTGGCAGGACCGGCGGCTGCGAGTGCTGAAACGGCGAACAAATCGAATGAGCT
TACAGCACCGTCGATCAAAAGCGGAACCATTCTTCATGCATGGAATTGGTCGTTC
AATACGTTAAAACACAATATGAAGGATATTCATGATGCAGGATATACAGCCATT
CAGACATCTCCGATTAACCAAGTAAAGGAAGGGAATCAAGGAGATAAAAGCATG
TCGAACTGGTACTGGCTGTATCAGCCGACATCGTATCAAATTGGCAACCGTTACT
TAGGTACTGAACAAGAATTTAAAGAAATGTGTGCAGCCGCTGAAGAATATGGCA
TAAAGGTCATTGTTGACGCGGTCATCAATCATACCACCAGTGATTATGCCGCGAT
TTCCAATGAGGTTAAGAGTATTCCAAACTGGACACATGGAAACACACAAATTAA
AAACTGGTCTGATCGGATCCTAGAAGCTTATCGAATTCGCGGCCGCTTCTAGAGC
AATACGCAAACCGCCTCTCCCCGCGCGTTGGCCGATTCATTAATGCAGCTGGCAC
GACAGGTTTCCCGACTGGAAAGCGGGCAGTGAGCGCAACGCAATTAATGTGAGT
TAGCTCACTCATTAGGCACCCCAGGCTTTACACTTTATGCTTCCGGCTCGTATGTT
GTGTGGAATTGTGAGCGGATAACAATTTCACACATACTAGAGAAAGAGGAGAAA
TACTAGATGGCTTCCTCCGAAGACGTTATCAAAGAGTTCATGCGTTTCAAAGTTC
GTATGGAAGGTTCCGTTAACGGTCACGAGTTCGAAATCGAAGGTGAAGGTGAAG
GTCGTCCGTACGAAGGTACCCAGACCGCTAAACTGAAAGTTACCAAAGGTGGTC
CGCTGCCGTTCGCTTGGGACATCCTGTCCCCGCAGTTCCAGTACGGTTCCAAAGC
TTACGTTAAACACCCGGCTGACATCCCGGACTACCTGAAACTGTCCTTCCCGGAA
GGTTTCAAATGGGAACGTGTTATGAACTTCGAAGACGGTGGTGTTGTTACCGTTA
CCCAGGACTCCTCCCTGCAAGACGGTGAGTTCATCTACAAAGTTAAACTGCGTGG
TACCAACTTCCCGTCCGATGGTCCGGTTATGCAGAAAAAAACCATGGGTTGGGA
AGCTTCCACCGAACGTATGTACCCGGAAGACGGTGCTCTGAAAGGTGAAATCAA
AATGCGTCTGAAACTGAAAGACGGTGGTCACTACGACGCTGAAGTTAAAACCAC
CTACATGGCTAAAAAACCGGTTCAGCTGCCGGGTGCTTACAAAACCGACATCAA
ACTGGACATCACCTCCCACAACGAAGACTACACCATCGTTGAACAGTACGAACG
TGCTGAAGGTCGTCACTCCACCGGTGCTTAATAACGCTGATAGTGCTAGTGTAGA
TCGCTACTAGAGCCAGGCATCAAATAAAACGAAAGGCTCAGTCGAAAGACTGGG
CCTTTCGTTTTATCTGTTGTTTGTCGGTGAACGCTCTCTACTAGAGTCACACTGGC
TCACCTTCGGGTGGGCCTTTCTGCGTTTATATACTAGTAGCGGCCGC
pBSOEXylRPxylA_{(V2)}
SEQ ID NO: 30
GGGTGCACGAGTGGGTTACATCGAACTGGATCTCAACAGCGGTAAGATCCTTGA
GAGTTTTCGCCCCGAAGAACGTTTTCCAATGATGAGCACTTTTAAAGTTCTGCTA
TGTGGCGCGGTATTATCCCGTATTGACGCCGGGCAAGAGCAACTCGGTCGCCGCA
TACACTATTCTCAGAATGACTTGGTTGAGTACTCACCAGTCACAGAAAAGCATCT
TACGGATGGCATGACAGTAAGAGAATTATGCAGTGCTGCCATAACCATGAGTGA
TAACACTGCGGCCAACTTACTTCTGACAACGATCGGAGGACCGAAGGAGCTAAC
CGCTTTTTTGCACAACATGGGGGATCATGTAACTCGCCTTGATCGTTGGGAACCG
GAGCTGAATGAAGCCATACCAAACGACGAGCGTGACACCACGATGCCTGTAGCA
ATGGCAACAACGTTGCGCAAACTATTAACTGGCGAACTACTTACTCTAGCTTCCC
GGCAACAATTAATAGACTGGATGGAGGCGGATAAAGTTGCAGGACCACTTCTGC
GCTCGGCCCTTCCGGCTGGCTGGTTTATTGCTGATAAATCTGGAGCCGGTGAGCG
TGGGTCTCGCGGTATCATTGCAGCACTGGGGCCAGATGGTAAGCCCTCCCGTATC
GTAGTTATCTACACGACGGGGAGTCAGGCAACTATGGATGAACGAAATAGACAG
ATCGCTGAGATAGGTGCCTCACTGATTAAGCATTGGTAACTGTCAGACCAAGTTT
ACTCATATATACTTTAGATTGATTTAAAACTTCATTTTTAATTTAAAAGGATCTAG
GTGAAGATCCTTTTTGATAATCTCATGACCAAAATCCCTTAACGTGAGTTTTCGTT
CCACTGAGCGTCAGACCCCGTAGAAAAGATCAAAGGATCTTCTTGAGATCCTTTT
TTTCTGCGCGTAATCTGCTGCTTGCAAACAAAAAAACCACCGCTACCAGCGGTGG
TTTGTTTGCCGGATCAAGAGCTACCAACTCTTTTTCCGAAGGTAACTGGCTTCAG
CAGAGCGCAGATACCAAATACTGTTCTTCTAGTGTAGCCGTAGTTAGGCCACCAC
TTCAAGAACTCTGTAGCACCGCCTACATACCTCGCTCTGCTAATCCTGTTACCAGT
GGCTGCTGCCAGTGGCGATAAGTCGTGTCTTACCGGGTTGGACTCAAGACGATAG
TTACCGGATAAGGCGCAGCGGTCGGGCTGAACGGGGGGTTCGTGCACACAGCCC
AGCTTGGAGCGAACGACCTACACCGAACTGAGATACCTACAGCGTGAGCTATGA
GAAAGCGCCACGCTTCCCGAAGGGAGAAAGGCGGACAGGTATCCGGTAAGCGG
CAGGGTCGGAACAGGAGAGCGCACGAGGGAGCTTCCAGGGGGAAACGCCTGGT
ATCTTTATAGTCCTGTCGGGTTTCGCCACCTCTGACTTGAGCGTCGATTTTTGTGA
TGCTCGTCAGGGGGGCGGAGCCTATGGAAAAACGCCAGCAACGCGGCCTTTTTA
CGGTTCCTGGCCTTTTGCTGGCCTTTTGCTCACATGTTCTTTCCTGCGTTATCCCCT
GATTCTGTGGATAACCGTATTACCGCCTTTGAGTGAGCTGATACCGCTCGCCGCA
GCCGAACGACCGAGCGCAGCGAGTCAGTGAGCGAGGAAGCGGAAGAGCGCCCA
ATACGCAAACCGCCTCTCCCCGCGCGTTGGCCGATTCATTAATGCAGCTGGCACG
ACAGGTTTCCCGACTGGAAAGCGGGCAGTGAGCGCAACGCAATTAATGTGAGTT
AGCTCACTCATTAGGCACCCCAGGCTTTACACTTTATGCTTCCGGCTCGTATGTTG
TGTGGAATTGTGAGCGGATAACAATTTCACACAGGAAACAGCTATGACCATGAT
TACGCCAAGCTTGCATGCCTGCAGCGGCCGCTACTAGTATATAAACGCAGAAAG
GCCCACCCGAAGGTGAGCCAGTGTGACTCTAGTAGAGAGCGTTCACCGACAAAC
AACAGATAAAACGAAAGGCCCAGTCTTTCGACTGAGCCTTTCGTTTTATTTGATG
CCTGGCTCTAGTAGCGATCTACACTAGCACTATCAGCGTTATTAAGCACCGGTGG
AGTGACGACCTTCAGCACGTTCGTACTGTTCAACGATGGTGTAGTCTTCGTTGTG
GGAGGTGATGTCCAGTTTGATGTCGGTTTTGTAAGCACCCGGCAGCTGAACCGGT
TTTTTAGCCATGTAGGTGGTTTTAACTTCAGCGTCGTAGTGACCACCGTCTTTCAG
TTTCAGACGCATTTTGATTTCACCTTTCAGAGCACCGTCTTCCGGGTACATACGTT
CGGTGGAAGCTTCCCAACCCATGGTTTTTTTCTGCATAACCGGACCGTCGGACGG
GAAGTTGGTACCACGCAGTTTAACTTTGTAGATGAACTCACCGTCTTGCAGGGAG
GAGTCCTGGGTAACGGTAACAACACCACCGTCTTCGAAGTTCATAACACGTTCCC
ATTTGAAACCTTCCGGGAAGGACAGTTTCAGGTAGTCCGGGATGTCAGCCGGGT
GTTTAACGTAAGCTTTGGAACCGTACTGGAACTGCGGGGACAGGATGTCCCAAG
CGAACGGCAGCGGACCACCTTTGGTAACTTTCAGTTTAGCGGTCTGGGTACCTTC
GTACGGACGACCTTCACCTTCACCTTCGATTTCGAACTCGTGACCGTTAACGGAA
CCTTCCATACGAACTTTGAAACGCATGAACTCTTTGATAACGTCTTCGGAGGAAG
CCATCTAGTATTTCTCCTCTTTCTCTAGTATGTGTGAAATTGTTATCCGCTCACAA
TTCCACACAACATACGAGCCGGAAGCATAAAGTGTAAAGCCTGGGGTGCCTAAT
GAGTGAGCTAACTCACATTAATTGCGTTGCGCTCACTGCCCGCTTTCCAGTCGGG
AAACCTGTCGTGCCAGCTGCATTAATGAATCGGCCAACGCGCGGGGAGAGG
CGGTTTGCGTATTGCTCTAGATTTTTTTTTGAATTCTACAGATGCATTTTATTTCATATAGTA
AGTACATCACCTATTAGTTTGTTGTTTAAACAAACTAACTTATTTTCATCTTA
TATAACCTCGTCAGTATTTTCAATATTTTTTTTAGTTTTTTATGAACACATTAGAT
ATAATAAAGGGAAGATTCGCTATGTACTATGTTGATACTTAATTTAAAGATTAAA
CAAATGGAGTGGATGAAGTGGATATCGCTGATCAAACCTTTGTCAAAAAAGTAA
ATCAAAAGTTATTATTAAAAGAAATCCTTAAAAATTCACCTATTTCAAGAGCAAA
ATTATCTGAAATGACTGGATTAAATAAATCAACTGTCTCATCACAGGTAAACACG
TTAATGAAAGAAAGTATGGTATTTGAAATAGGTCAAGGACAATCAAGTGGCGGA
AGAAGACCTGTCATGCTTGTTTTTAATAAAAAGGCAGGATACTCCGTTGGAATAG
ATGTTGGTGTGGATTATATTAATGGCATTTTAACAGACCTTGAAGGAACAATCGT
TCTTGATCAATACCGCCATTTGGAATCCAATTCTCCAGAAATAACGAAAGACATT
TTGATTGATATGATTCATCACTTTATTACGCAAATGCCCCAATCTCCGTACGGGCT
TATTGGTATAGGTATTTGCGTGCCTGGACTCATTGATAAAGATCAAAAAATTGTT
TTCACTCCGAACTCCAACTGGAGAGATATTGACTTAAAATCTTCGATACAAGAGA
AGTACAATGTGCCTGTTTTTATTGAAAATGAGGCAAATGCTGGCGCATATGGAGA
AAAAGTATTTGGAGCTGCAAAAAATCACGATAACATTATTTACGTAAGTATCAGC
ACAGGAATAGGGATCGGTGTTATTATCAACAATCATTTATATAGAGGAGTAAGC
GGCTTCTCTGGAGAAATGGGACATATGACAATAGACTTTAATGGTCCTAAATGCA
GTTGCGGAAACCGAGGATGCTGGGAATTGTATGCTTCAGAGAAGGCTTTATTAA
AATCTCTTCAGACCAAAGAGAAAAAACTGTCCTATCAAGATATCATAAACCTCGC
CCATCTGAATGATATCGGAACCTTAAATGCATTACAAAATTTTGGATTCTATTTA
GGAATAGGCCTTACCAATATTCTAAATACTTTCAACCCACAAGCCGTAATTTTAA
GAAATAGCATAATTGAATCGCATCCTATGGTTTTAAATTCAATGAGAAGTGAAGT
ATCATCAAGGGTTTATTCCCAATTAGGCAATAGCTATGAATTATTGCCATCTTCCT
TAGGACAGAATGCACCGGCATTAGGAATGTCCTCCATTGTGATTGATCATTTTCT
GGACATGATTACAATGTAATTTTTTATGGAATGGACAGCTCATCTTTAAAGATGA
GTTTTTTTATTCTAGGAGTATTTCTGAAGCAATAGTGACATGGCACCTTCTCATAT
GAAAAAGGAGTTCTAAAATAGAAATCTCCTTTTTCATGTGCAAATTATTTTTCTTT
ATAACGAAAATATCTAAAGTCGGCCAATTCACTGGCCGTCGTTTTACAACGTCGT
GACTGGGAAAACCCTGGCGTTACCCAACTTAATCGCCTTGCAGCACATCCCCCTT
TCGCCAGCTGGCGTAATAGCGAAGAGGCCCGCACCGATCGCCCTTCCCAACAGTT
GCGCAGCCTGAATGGCGAATGGCGCCTGATGCGGTATTTTCTCCTTACGCATCTG
TGCGGTATTTCACACCGCATATGGTGCACTCTCAGTACAATCTGCTCTGATGCCG
CATAGTTAAGCCAGCCCCGACACCCGCCAACACCCGCTGACGCGCCCTGACGGG
CTTGTCTGCTCCCGGCATCCGCTTACAGACAAGCTGTGACCGTCTCCGGGAGCTG
CATGTGTCAGAGGTTTTCACCGTCATCACCGAAACGCGCGAGACGAAAGGGCCT
CGTGATACGCCTATTTTTATAGGTTAATGTCATGATAATAATGGTTTCTTAGACGT
CAGGTGGCACTTTTCGGGGAAATGTGCGCGGAACCCCTATTTGTTTATTTTTCTAA
ATACATTCAAATATGTATCCGCTCATGAGACAATAACCCTGATAAATGCTTCAAT
AATCCATCCTCCAAAGTTGGAGAGTGAGTTTTATGTCGCAAATATTAATGTTTCT
GGTGAACCTTATCAAATTTTCGTTGATTTAATAGAAACATAGCGGTAAAATTAGC
AGTAACTTAATAGAACGGAAATGAAAAAAGCCACTCTCATATGCTATTGGCTAC
CAACCTTTAGCGAGAATGACTTAATCCTGTACAGCCATACAGGACTTCGACTTAT
AAGAGGCGCCAACTTCAAATAAGTTATTTGCCTTGTTTTCGCGAACAAGGCTTAT
TAGATACACCTATTGTACCGTTACTCTACGAATATTTCAAGTAGTAATTACTAGC
ATTGTCCGTTACTCTACGAATATTTCAAGTAGTAATTACTAGCATTGTCCGTTACT
CTACGAATATTTCAAGTAGTAATTACTAGCATTGTCATATACATAATAAAACGGA
TATAAAAGGGCGTTTTCTATACCTAGAAGTCTTGTAAATGTACAGGGCGTTTAGA
TATAGAGAACGCCCTTTTTGTGTTCCGTTCCAGTGGAAGCTACCACTTTAAAAAG
ATGGTCTAGTGTAGCCAATGCAGGAGAGTACACTCGGATATCAGTTGTCGTTGCA
TTCAACTGTCTGACGTAAGCGAGGTAAAGGACACAAGCCTTGCATAAAACAAGC
CTACGGGATGTAAATCCTAATAATGATGATAACCAAGACGTTAGCGGCAAAAAG
TGTTGGGGGTTCAAAATAAGACATGATTGTGCGACTGGAGTTAAACAGTTACTCG
TAAGCGGCGATCATGACACTGATTCACGGCTATTCTTGTACAAGCTAGCTTTATT
ACAAGGATATGCGGGTTATATAGCGAATCACCCGAAAGGGAACGGTGTTGGGCG
TGAGAAACGCACCGTACGGCGCAATACAATGCCAATAAGCTATATACGGACGGT
ATAGTAGTTTTGTAAGCTATAACCGTTTGTCGTCAATGCAACCAATCTCAATTCG
AGACCTCGGCATCTAAGCCAGTACGAATGAGTGGGCGTTTTAACCTCGTAAATTT
TCAACAGGGGTTACTATGCCCAAAACTACATTCAGATTTCCTAACAAACTCGCCA
GTATGAAAACCTTAAGACCTTAAAGTCAAGGGATTTGAAGGATTTTAACCTCGAT
TAGCAAAAAATGTAGAGTACTGAAGCAACTACCATTAACTAAGATAGTGGGGGA
TTGAGGAAGAATCCAGAGCTGTTTAAATCAAGTGAAAGACAAGATGAAATTAAA
AGAATAGTGAAAGATAGGGGAGTGGTTCTCTATGAGAAAGGAAATGGCTAGAGA
ACAAAGGCAGCGGTTTATTGATCTATTGTTAGACTTTATGGTAAAGAATCCTCAT
TTATTTGTTAATGGTACAGAGGATGAAAGTAATAATGTTGTTACAAAATGTAATA
GTGATATTAAAGAGGTTGCGGAGTCATATTTAACTCTTTTATAGTGAGAGGGTTA
AAACTAATTAATATGTATTAAGGCCCAATGTTGGAATTATTGTATTTCACTAGGC
AACCTACTTACTAAAAGTAAGATTATCCATTAGTGGATGTTATAATATTGGGTTT
TTTAACACAATAATCATCGCCTTTCGGTGTCGTTTGATAGAAAAGTAACCATTAG
CGATGAAAAAGTCAATATAAAAAGCCATCCGTAAAAAACGGATGGCTTACCGTA
CATAGGATCGTTGGTAGGGCGGCGTATCCTACATCTCTGGTAACTTACCTAGCCA
ATCAAATGCTTGAGAACGGCGGTTAGATAAGCGCGTGGGGAACCTTTCCCACCTC
AAAGATCCTATATCATTATTATGTTACTTTCTACAGGTAGTATACCATGTTCTTAT
ATTTTAGTAAACTCCCCGTTAGCTTAACAGGTCTTTGTAAGCAATTAAACGTCCA
CTATTCAATCGTCTTTGGATTTTCGCAGGACCGTTTTTTAGATCGAACATAGTTGA
TAAGAACAAATAACCGCTTGGGTCCAACTTTATAGCAATTAGTATATGGTCATTT
AAAATCTTTACCAATTCAACGCTATTAGGTTCTTTAGGATTTTGCCCGACATAGTC
GGGGTGTTCAACGATATCTTTTATGTGCGATGAATATTTTTCATAAATACCAGGA
TGTTGTTTCTTTACGTGCTTTATAAATCCGGGAAACATTTTTACATCGTTAGAAGT
GCAAGTCAAGTTATATGTATCTATAATGATTTGTGGAAGTTTTGCCACAACAGTT
GGTTTATTTACAATCTTTTTTTTATTAGCCGTCAAATTTCTCCCTCATCTCGTCTCT
TTATATCTTTATTTTATCATAAAGGAGTATTTGAACCGTCGCGCGGGACAGGTTT
ATGATAGGGATATTTTATTGAATAATTGATGGTATAAGGGACTTTCATGCTTGGA
AAGTGGGGATTATGAATTAGATGCTTGTCCACAATATGTTCCAATGTAATTAAAA
TTTATGTTCCCACCTTGACCAAACATCACGTCCATACTTAAATCGTCCCTCCTTTA
ATAGGTAAAATATTAATTTACCTTAATAAAAAAATAATGGATAATAGTATTCGTC
TGAATTTATATAATCAGGGGGAACTATTGATGCTGGGGATACTATTTACAGCGGC
GCCATCTACTGATGTCGTAAAGGATTTGCAAGATAAAGTTATATCATTGCAGGAT
CATGAGGTAGCGTTTTTGAACACCACGATATCTAATATGTTGATCCCCGAAGCAA
ACTTAAGAGTGTGTTGATAGTGCAGTATCTTAAAATTTTGTGTATAATAGGAATT
GAAGTTAAATTAGATGCTAAAAATTTGTAATTAAGAAGGAGGGATTCGTCATGTT
GGTATTCCAAATGCGTAATGTAGATAAAACATCTACTGTTTTGAAACAGACTAAA
AACAGTGATTACGCAGATAAATAAATACGTTAGATTAATTCCTACCAGTGACTAA
TCTTATGACTTTTTAAACAGATAACTAAAATTACAAACAAATCGTTTAACTTCTGT
ATTTATTTACAGATGTAATCACTTCAGGAGTAATTACATGAACAAAAATATAAAA
TATTCTCAAAACTTTTTAACGAGTGAAAAAGTACTCAACCAAATAATAAAACAAT
TGAATTTAAAAGAAACCGATACCGTTTACGAAATTGGAACAGGTAAAGGGCATT
TAACGACGAAACTGGCTAAAATAAGTAAACAGGTAACGTCTATTGAATTAGACA
GTCATCTATTCAACTTATCGTCAGAAAAATTAAAACTGAACATTCGTGTCACTTT
AATTCACCAAGATATTCTACAGTTTCAATTCCCTAACAAACAGAGGTATAAAATT
GTTGGGAGTATTCCTTACCATTTAAGCACACAAATTATTAAAAAAGTGGTTTTTG
AAAGCCATGCGTCTGACATCTATCTGATTGTTGAAGAAGGATTCTACAAGCGTAC
CTTGGATATTCACCGAACACTAGGGTTGCTCTTGCACACTCAAGTCTCGATTCAG
CAATTGCTTAAGCTGCCAGCGGAATGCTTTCATCCTAAACCAAAAGTAAACAGTG
TCTTAATAAAACTTACCCGCCATACCACAGATGTTCCAGATAAATATTGGAAGCT
ATATACGTACTTTGTTTCAAAATGGGTCAATCGAGAATATCGTCAACTGTTTACT
AAAAATCAGTTTCATCAAGCAATGAAACACGCCAAAGTAAACAATTTAAGTACC
ATTACTTATGAGCAAGTATTGTCTATTTTTAATAGTTATCTATTATTTAACGGGAG
GAAATAATTCTATGAGTCGCTTTTTTAAATTTGGAAAGTTACACGTTACTAAAGG
GAATGGAGATAAATTATTAGATATACTACTGACAGCTTCCAAGAAGCTAAAGAG
GTCCCTAGCGCCTACGGGGAATTTGGGGTACATTGAAAAAGGAAGAGTATGAGT
ATTCAACATTTCCGTGTCGCCCTTATTCCCTTTTTTGCGGCATTTTGCCTTCCTGTT
TTTGCTCACCCAGAAACGCTGGTGAAAGTAAAAGATGCTGAAGATCAGTT
agrBD loci Group I
SEQ ID NO: 31
TTGAATTATTTTGATAATAAAATTGACCAGTTTGCCACGTATCTTCAAAAGAGAA
ATAACTTAGATCATATTCAATTTTTGCAAGTACGATTAGGGATGCAGGTCTTAGC
TAAAAATATAGGTAAATTAATTGTTATGTATACTATTGCCTATATTTTAAACATTT
TTCTGTTTACGTTAATTACGAATTTAACATTTTATTTAATAAGAAGACATGCACAT
GGTGCACATGCACCTTCTTCTTTTTGGTGTTATGTAGAAAGTATTATACTATTTAT
ACTTTTACCTTTAGTAATAGTAAATTTTCATATTAACTTTTTAATTATGATTATTTT
AACAGTTATTTCTTTAGGTGTAATCTCAGTATATGCTCCTGCAGCAACTAAAAAG
AAGCCCATTCCTGTGCGACTTATTAAACGAAAAAAATATTATGCGATTATTGTTA
GTTTAACCCTTTTCATTATCACACTTATCATCAAAGAGCCATTTGCCCAATTCATT
CAATTAGGCATCATAATAGAAGCTATTACATTATTACCTATTTTCTTTATTAAGGA
GGACTTAAAATGAATACATTATTTAACTTATTTTTTGATTTTATTACTGGGATTTT
AAAAAACATTGGTAACATCGCAGCTTATAGTACTTGTGACTTCATAATGGATGAA
GTTGAAGTACCAAAAGAATTAACACAATTACACGAATAA
agrBD loci Group III
SEQ ID NO: 32
TTGAATTATTTTGATAACAAAATTGACCAGTTTGCCACGTATCTTCAAAAGAGAA
ATAACTTAGATCATATTCAATTTTTGCAAGTACGATTAGGGATGCAGGTCTTAGC
TAAAAATATAGGAAAACTGATTGTTATGTATACTCTTGCCTATATTTTAAACATTT
TTATCTTCACTTTAATTACTAATATATCGTTTTATCTAATTAGAAGGTATGCACAT
GGTGCACATGCACCTTCATCTTTTTGGTGTTATATAGAAAGCATTACACTTTTTAT
TGTTCTACCTCTATTAGTACTACATTTTCATATTAATGAAACACTAATGATGTTTT
TAGCATTATTAAGTGTTGGCGTAGTGATTAAATATGCACCTGCAGCTACTAAGAA
GAAGCCTATCCCGGCAAGGCTTGTAAAGCAAAAAAGATATTTTTCAATTATAATT
AGTACAATTCTTTTCATTATCACACTTTTTGTAAAGGAACCATATACCCAATTTAT
TCAACTGGGTATTATTATACAAGCTATTACATTACTACCAATATACTATTCAAAG
GAGGACTAAATTATGAAAAAATTACTCAACAAAGTTATTGAGCTTTTAGTTGACT
TTTTCAACAGCATCGGTTACAGAGCAGCCTATATAAATTGTGATTTTTTATTGGAT
GAAGCTGAAGTACCAAAAGAATTAACTCAATTACACGAATAA
PliaG promoter
SEQ ID NO: 33
CTAGTATCATTCATTCTATTATAAAGGAAAAGCTTGCGAATTGATACGTGCGG
AAGGGAAGGACTTGACCGCAAATCCTTCCCCGTTCCGCTTATTCATTTGCCGCTTTTGTC
TGGTCTGATTTTTGCTCTAGAAGCGGCCGCGAATTC
PlepA promoter
SEQ ID NO: 34
CTAGAGAGTCAATGTATGAATGGATACGGGATATGAATCAATAAGTACGTGAAA
GAGAAAAGCAACCCAGATATGATAGGGAACTTTTCTCTTTCTTGTTTTACATTGA
ATCTTTACAATCCTATTGATATAATCTAAGCTAGTGTATTTTGCGTTTAATAGTTA
CTAG
Pveg promoter
SEQ ID NO: 35
CTAGAGGGAGTTCTGAGAATTGGTATGCCTTATAAGTCCAATTAACAGTTGAAAA
CCTGCATAGGAGAGCTATGCGGGTTTTTTATTTTACATAATGATACATAATTTACC
GAAACTTGCGGAACATAATTGAGGAATCATAGAATTTTGTCAAAATAATTTTATT
GACAACGTCTTATTAACGTTGATATAATTTAAATTTTATTTGACAAAAATGGGCT
CGTGTTGTACAATAAATGTAGTTACTAG
mroQ gene
SEQ ID NO: 36
ATGACAAGATTATGGGCATCATTGCTAACTGTTATTATTTATATATTGTCTCAATT
TTTACCGCTTCTCATTGTAAAAAAATTACCATTTGTACAATATAGTGGCATAGAA
CTGACTAAAGCAGTCATTTACATACAACTTGTTCTATTTTTAATCGCCGCCACGAC
GATTATTTTAATTAATTTAAAAATTAAAAATCCAACAAAATTAGAATTAGAAGTT
AAAGAACCTAAAAAATATATCATTCCATGGGCATTGCTTGGATTTGCATTGGTAA
TGATTTATCAAATGGTAGTGAGCATTGTATTAACGCAAATTTATGGTGGACAACA
AGTAAGTCCTAATACAGAAAAGCTAATTATTATTGCTCGAAAAATACCTATATTT
ATCTTCTTTGTATCTATTATTGGTCCTTTATTAGAAGAATATGTATTCAGAAAAGT
AATCTTTGGAGAATTATTTAATGCGATTAAAGGTAATCGTATCGTGGCATTTATT
ATTGCTACAACAGTAAGTTCATTAATATTTGCATTAGCACATAATGATTTCAAAT
TTATTCCAGTTTATTTTGGTATGGGTGTCATTTTTTCATTAGCATATGTTTGGACA
AAACGGCTTGCTGTTCCAATTATTATCCATATGTTACAAAACGGATTTGTCGTTAT
ATTCCAATTACTTAATCCTGAGGCTTTGAAAAAAGCCACGGAACAAGCGAATTTT
ATATATCACATTTTTATTCCATAA
agrB gene Group I
SEQ ID NO: 37
TTGAATTATTTTGATAATAAAATTGACCAGTTTGCCACGTATCTTCAAAAGAGAA
ATAACTTAGATCATATTCAATTTTTGCAAGTACGATTAGGGATGCAGGTCTTAGC
TAAAAATATAGGTAAATTAATTGTTATGTATACTATTGCCTATATTTTAAACATTT
TTCTGTTTACGTTAATTACGAATTTAACATTTTATTTAATAAGAAGACATGCACAT
GGTGCACATGCACCTTCTTCTTTTTGGTGTTATGTAGAAAGTATTATACTATTT
ATACTTTTACCTTTAGTAATAGTAAATTTTCATATTAACTTTTTAATTATGATTATTTTAA
CAGTTATTTCTTTAGGTGTAATCTCAGTATATGCTCCTGCAGCAACTAAAAAG
AAGCCCATTCCTGTGCGACTTATTAAACGAAAAAAATATTATGCGATTATTGTTA
GTTTAACCCTTTTCATTATCACACTTATCATCAAAGAGCCATTTGCCCAATTCATT
CAATTAGGCATCATAATAGAAGCTATTACATTATTACCTATTTTCTTTATTAAGGA
GGACTTAAAATGA
agrB Group III
SEQ ID NO: 38
TTGAATTATTTTGATAACAAAATTGACCAGTTTGCCACGTATCTTCAAAAGAGAA
ATAACTTAGATCATATTCAATTTTTGCAAGTACGATTAGGGATGCAGGTCTTAGC
TAAAAATATAGGAAAACTGATTGTTATGTATACTCTTGCCTATATTTTAAACATTT
TTATCTTCACTTTAATTACTAATATATCGTTTTATCTAATTAGAAGGTATGCACAT
GGTGCACATGCACCTTCATCTTTTTGGTGTTATATAGAAAGCATTACACTTTTTAT
TGTTCTACCTCTATTAGTACTACATTTTCATATTAATGAAACACTAATGATGTTTT
TAGCATTATTAAGTGTTGGCGTAGTGATTAAATATGCACCTGCAGCTACTAAGAA
GAAGCCTATCCCGGCAAGGCTTGTAAAGCAAAAAAGATATTTTTCAATTATAATT
AGTACAATTCTTTTCATTATCACACTTTTTGTAAAGGAACCATATACCCAATTTAT
TCAACTGGGTATTATTATACAAGCTATTACATTACTACCAATATACTATTCAAAG
GAGGACTA
agrD gene Group I
SEQ ID NO: 39
ATGAATACATTATTTAACTTATTTTTTGATTTTATTACTGGGATTTTAAAAAACAT
TGGTAACATCGCAGCTTATAGTACTTGTGACTTCATAATGGATGAAGTTGAAGTA
CCAAAAGAATTAACACAATTACACGAATAA
agrD gene Group III
SEQ ID NO: 40
ATGAAAAAATTACTCAACAAAGTTATTGAGCTTTTAGTTGACTTTTTCAACAGCA
TCGGTTACAGAGCAGCCTATATAAATTGTGATTTTTTATTGGATGAAGCTGAAGT
ACCAAAAGAATTAACTCAATTACACGAATAA
agrD_D29A Group I
SEQ ID NO: 41
ATGAATACATTATTTAACTTATTTTTTGATTTTATTACTGGGATTTTAAAAAACAT
TGGTAACATCGCAGCTTATAGTACTTGTGCCTTCATAATGGATGAAGTTGAAGTA
CCAAAAGAATTAACACAATTACACGAATAA
agrD_D29A Group III
SEQ ID NO: 42
ATGAAAAAATTACTCAACAAAGTTATTGAGCTTTTAGTTGACTTTTTCAACAGCA
TCGGTTACAGAGCAGCCTATATAAATTGTGCTTTTTTATTGGATGAAGCTGAAGT
ACCAAAAGAATTAACTCAATTACACGAATAA
Terminator stem sequence
SEQ ID NO: 43
GATACGCACCCATTAGGGTGCGAAAAAAA
Spacer sequence
CTGACTA
Ribosome-binding sequence
AAGGAGG

### Equivalents

While certain embodiments have been illustrated and described, a person with ordinary skill in the art, after reading the foregoing specification, can effect changes, substitutions of equivalents and other types of alterations to the nanoparticles of the present technology or derivatives, prodrugs, or pharmaceutical compositions thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, conjugates, reagents, compounds, compositions, labeled compounds or biological systems, which can, of course, vary. All methods described herein can be performed in any suitable order unless otherwise indicated herein or otherwise clearly contradicted by context. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof. No language in the specification should be construed as indicating any non-claimed element as essential.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Likewise, the use of the terms “comprising,” “including,” “containing,” etc. shall be understood to disclose embodiments using the terms “consisting essentially of” and “consisting of.” The phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the technology. This includes the generic description of the technology with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member, and each separate value is incorporated into the specification as if it were individually recited herein.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims, along with the full scope of equivalents to which such claims are entitled.

### References

1 Tal-Gan, Y., Stacy, D. M., Foegen, M. K., Koenig, D. W. & Blackwell, H. E. Highly potent inhibitors of quorum sensing in Staphylococcus aureus revealed through a systematic synthetic study of the group-III autoinducing peptide. J Am Chem Soc 135, 7869-7882, doi:10.1021/ja3112115 (2013).
2 Biedendieck, R. et al. Systems biology of recombinant protein production using Bacillus megaterium. Methods Enzymol 500, 165-195, doi:10.1016/B978-0-12-385118-5.00010-4 (2011).
3 Popp, P. F., Dotzler, M., Radeck, J., Bartels, J. & Mascher, T. The Bacillus BioBrick Box 2.0: expanding the genetic toolbox for the standardized work with Bacillus subtilis. Sci Rep 7, 15058, doi:10.1038/s41598-017-15107-z (2017).
4 Radeck, J. et al. The Bacillus BioBrick Box: generation and evaluation of essential genetic building blocks for standardized work with Bacillus subtilis. J Biol Eng 7, 29, doi:10.1186/1754-1611-7-29 (2013).
5 Bennallack, P. R., Burt, S. R., Heder, M. J., Robison, R. A. & Griffitts, J. S. Characterization of a novel plasmid-borne thiopeptide gene cluster in Staphylococcus epidermidis strain 115. J Bacteriol 196, 4344-4350, doi:10.1128/JB.02243-14 (2014).
6 Yarwood, J. M., Bartels, D. J., Volper, E. M. & Greenberg, E. P. Quorum sensing in Staphylococcus aureus biofilms. J Bacteriol 186, 1838-1850, doi:10.1128/JB.186.6.1838-1850.2004 (2004).
7 Kirchdoerfer, R. N. et al. Structural basis for ligand recognition and discrimination of a quorum-quenching antibody. J Biol Chem 286, 17351-17358, doi:10.1074/jbc.M111.231258 (2011).
8 Malone, C. L., Boles, B. R. & Horswill, A. R. Biosynthesis of Staphylococcus aureus autoinducing peptides by using the synechocystis DnaB mini-intein. Appl Environ Microbiol 73, 6036-6044, doi:10.1128/AEM.00912-07 (2007).
9 Peng, H. L., Novick, R. P., Kreiswirth, B., Kornblum, J. & Schlievert, P. Cloning, characterization, and sequencing of an accessory gene regulator (agr) in Staphylococcus aureus. J Bacteriol 170, 4365-4372, doi:10.1128/jb.170.9.4365-4372.1988 (1988).
10 Ji, G., Beavis, R. & Novick, R. P. Bacterial interference caused by autoinducing peptide variants. Science 276, 2027-2030, doi:10.1126/science.276.5321.2027 (1997).

1. An engineered bacterial cell capable of producing a non-native autoinducing peptide (AIP) analog that inhibits the accessory gene regulator (agr) quorum sensing (QS) system, wherein the engineered bacterial cell is transformed with at least one plasmid comprising one or more nucleotide sequences encoding one or more of agrBD loci, mroQ gene, argB gene, or argD gene, and one or more promoters.

2. The engineered bacterial cell of claim 1, wherein the engineered bacterial cell is an engineered Gram-positive bacterium.

3. The engineered bacterial cell of claim 2, wherein the Gram-positive bacterium is selected from B. subtilis strain or P. megaterium strain.

4. The engineered bacterial cell of claim 3, wherein the B. subtilis strain is Bs-ΔydiL.

5. The engineered bacterial cell of claim 4, wherein the one or more promoters are selected from the group consisting of PliaG promoter, PlepA promoter, and Pveg promoter.

6. The engineered bacterial cell of claim 5, wherein the plasmid comprises at least the nucleotide sequence encoding the PliaG promoter and the mroQ gene.

7. The engineered bacterial cell of claim 6, further comprising a second plasmid comprising a nucleotide sequence encoding at least the agrBD loci.

8. The engineered bacterial cell of claim 7, wherein the argBD loci is amplified from wild type S. aureus.

9. The engineered bacterial cell of claim 8, wherein the wild type S. aureuscomprises a group I or group III type arg QS system.

10. The engineered bacterial cell of claim 9, wherein the plasmid further comprises a ribosome-binding sequence of AAGGAGG and one or more of 7-nucleotide spacer inserted 5′ of each coding gene.

11. The engineered bacterial cell of claim 10, wherein the nucleotide sequence 3′ of the argD gene further comprises a terminator stem sequence.

12. (canceled)

13. The engineered bacterial cell of claim 11, wherein the argD gene comprises the mutation D29A.

14. The engineered bacterial cell of claim 13, wherein the argD gene is encoded by a nucleic acid sequence selected from SEQ ID NOs: 41 or 42.

15. The engineered bacterial cell of claim 14, wherein the bacterial cell is an inducible bacterial strain or a constitutive strain.

16. The engineered bacterial cell of claim 15, wherein the agr QS system is an S. aureus agr QS system.

17. An engineered bacterial cell transformed with one or more of:
(a) a plasmid comprising a nucleic acid sequence encoding a agrBD locus amplified from wild type S. aureus;
(b) a first plasmid comprising a nucleic acid sequence encoding a agrBD locus amplified from wild type S. aureus and a second plasmid comprising a nucleic acid sequence encoding a PliaG promoter and a mroQ gene; or
(c) a plasmid comprising a nucleic acid sequence encoding:
(i) a promoter selected from PliaG promoter, PlepA promoter, or Pveg promoter;
(ii) a mroQ gene, an argB gene, and an argD gene; and
(iii) a ribosome-binding sequence of AAGGAGG and one or more of 7-nucleotide spacer inserted 5′ of each coding gene.

18. (canceled)

19. The engineered bacterial cell of claim 17, wherein the argD gene is mutated and encoded by a nucleic acid sequence selected from SEQ ID NOs: 41 or 42.

20. (canceled)

21. A plasmid comprising one or more of the nucleic acid sequences of claim 17, wherein the argD gene is mutated and encoded by a nucleic acid sequence selected from SEQ ID NOs: 41 or 42.

22. A method of producing a non-native autoinducing peptide (AIP) analog comprising, transforming a bacterial cell with one or more of:
(a) a plasmid comprising a nucleic acid sequence encoding a agrBD locus amplified from wild type S. aureus;
(b) a first plasmid comprising a nucleic acid sequence encoding a agrBD locus amplified from wild type S. aureus and a second plasmid comprising a nucleic acid sequence encoding a PliaG promoter and a mroQ gene; or
(c) a plasmid comprising a nucleic acid sequence encoding:
(i) a promoter selected from PliaG promoter, PlepA promoter, or Pveg promoter;
(ii) a mroQ gene, an argB gene, and a mutated argD gene; and
(iii) a ribosome-binding sequence of AAGGAGG and one or more of 7-nucleotide spacer inserted 5′ of each coding gene.

23. The method of claim 22, wherein the AIP analog is an inhibitor of S. aureus agr quorum sensing (QS) system.

24. (canceled)

25. (canceled)


SEQUENCE LISTING (43 sequences)

SEQ ID NO: 1 - DNA, 42 bp, synthetic construct
  source 1..42: mol_type = other DNA
actgacactagtatgaattattttgataataaaattgaccag

SEQ ID NO: 2 - DNA, 29 bp, synthetic construct
  source 1..29: mol_type = other DNA
cgaattggatcccacctactatcacactc

SEQ ID NO: 3 - DNA, 42 bp, synthetic construct
  source 1..42: mol_type = other DNA
actgacactagtatgaattattttgataacaaaattgaccag

SEQ ID NO: 4 - DNA, 38 bp, synthetic construct
  source 1..38: mol_type = other DNA
cgaattggatcccacactttctcttatagttattcgtg

SEQ ID NO: 5 - DNA, 41 bp, synthetic construct
  source 1..41: mol_type = other DNA
caacttcatccattatgaaggcacaagtactataagctgcg

SEQ ID NO: 6 - DNA, 41 bp, synthetic construct
  source 1..41: mol_type = other DNA
cgcagcttatagtacttgtgccttcataatggatgaagttg

SEQ ID NO: 7 - DNA, 47 bp, synthetic construct
  source 1..47: mol_type = other DNA
cttcagcttcatccaataaaaaagcacaatttatataggctgctctg

SEQ ID NO: 8 - DNA, 47 bp, synthetic construct
  source 1..47: mol_type = other DNA
cagagcagcctatataaattgtgcttttttattggatgaagctgaag

SEQ ID NO: 9 - DNA, 56 bp, synthetic construct
  source 1..56: mol_type = other DNA
cgaatttctagaaaggaggctgactaatgaattattttgataataaaattgaccag

SEQ ID NO: 10 - DNA, 28 bp, synthetic construct
  source 1..28: mol_type = other DNA
agcagatgcatcacctactatcacactc

SEQ ID NO: 11 - DNA, 56 bp, synthetic construct
  source 1..56: mol_type = other DNA
cgaatttctagaaaggaggctgactaatgaattattttgataacaaaattgaccag

SEQ ID NO: 12 - DNA, 37 bp, synthetic construct
  source 1..37: mol_type = other DNA
agcagatgcatcacactttctcttatagttattcgtg

SEQ ID NO: 13 - DNA, 51 bp, synthetic construct
  source 1..51: mol_type = other DNA
cgaatttctagaaaggaggctgactaatgacaagattatgggcatcattgc

SEQ ID NO: 14 - DNA, 90 bp, synthetic construct
  source 1..90: mol_type = other DNA
ggactctgcagcggccgctactagtagctttttttcgcaccctaatgggtgcgtatcttatggaataaaaatgtgatatataaaattcgc

SEQ ID NO: 15 - DNA, 61 bp, synthetic construct
  source 1..61: mol_type = other DNA
ggactctgcagcggccgctactagtagcttatggaataaaaatgtgatatataaaattcgc

SEQ ID NO: 16 - DNA, 49 bp, synthetic construct
  source 1..49: mol_type = other DNA
gtgtaatctcagtatatgctcctgctgcaactaaaaagaagcccattcc

SEQ ID NO: 17 - DNA, 49 bp, synthetic construct
  source 1..49: mol_type = other DNA
ggaatgggcttctttttagttgcagcaggagcatatactgagattacac

SEQ ID NO: 18 - DNA, 54 bp, synthetic construct
  source 1..54: mol_type = other DNA
gtgttggcgtagtgattaaatatgcacctgctgctactaagaagaagcctatcc

SEQ ID NO: 19 - DNA, 54 bp, synthetic construct
  source 1..54: mol_type = other DNA
ggataggcttcttcttagtagcagcaggtgcatatttaatcactacgccaacac

SEQ ID NO: 20 - DNA, 71 bp, synthetic construct
  source 1..71: mol_type = other DNA
gatccgaattcgcggccgcttctagagaaggaggctgactaatgaattattttgataataaaattgaccag

SEQ ID NO: 21 - DNA, 51 bp, synthetic construct
  source 1..51: mol_type = other DNA
ggactctgcagcggccgctactagtatcattttaagtcctccttaataaag

SEQ ID NO: 22 - DNA, 71 bp, synthetic construct
  source 1..71: mol_type = other DNA
gatccgaattcgcggccgcttctagagaaggaggctgactaatgaattattttgataacaaaattgaccag

SEQ ID NO: 23 - DNA, 52 bp, synthetic construct
  source 1..52: mol_type = other DNA
ggactctgcagcggccgctactagtatagtcctcctttgaatagtatattgg

SEQ ID NO: 24 - DNA, 69 bp, synthetic construct
  source 1..69: mol_type = other DNA
gatccgaattcgcggccgcttctagagaaggaggctgactaatgaatacattatttaacttattttttg

SEQ ID NO: 25 - DNA, 72 bp, synthetic construct
  source 1..72: mol_type = other DNA
ggactctgcagcggccgctactagtatttttttcgcaccctaatgggtgcgtatccacctactatcacactc

SEQ ID NO: 26 - DNA, 71 bp, synthetic construct
  source 1..71: mol_type = other DNA
gatccgaattcgcggccgcttctagagaaggaggctgactaatgaaaaaattactcaacaaagttattgag

SEQ ID NO: 27 - DNA, 81 bp, synthetic construct
  source 1..81: mol_type = other DNA
ggactctgcagcggccgctactagtatttttttcgcaccctaatgggtgcgtatccacactttctcttatagttattcgtg

SEQ ID NO: 28 - DNA, 8813 bp, synthetic construct
  source 1..8813: mol_type = other DNA
tgtcacggaggttcaagttacctttagccggaagtgctggcattttgtccaattgagactcgtgcaactggtcagcgaactggtcgtagaaatcagccagtacatcacaagactcatatgtgtcaaccatagtttcgcgcactgctttgaacaggttcgcagcgtcagccggaatggtaccgaaggagtcgtgaatcagtgcaaaagattcgattccgtacttctcgtgtgcccacactacagtcttacgaaggtggctaccgtcttggctgtgtacaaagttaggagcgataccagactcctgtttgtgtgcatcaatctcgctatctttgttggtgttaatggtaggctgtaagcggaactgaccgaggaacatcaggttcaagcgcgtctgaataggcttcttgtattcctgccacacagggaaaccatcaggagttacccaatgcacagcgcaacgcttgcgaagaatctctccagtcttcttatctttgacctcagcagccagcagcttagcagcagacttaagccagttcattgcttcaaccgcagctaccaccgtcacgctcacagattcccaaatcagcttagccatgtatccagcagcctgattcggctgagtgaacatcagacccttgccggaatcaatagctggctgaatggtatcttccagcacttgttgacggaagccgaactctttggacccgtaagccagcgtcatgactgaacgcttagtcacactgcgagtaacaccgtaagccagccattgaccagccagtgccttagtgcccagcttgactttctcagagatttcaccagtgttctcatcggtcacggtaactacttcgttatcggtcccattgattgcgtctgcttgtagaatctcgttgactttcttagcaacaatcccgtagatgtcctgaacggtttcactaggaagcaagttaaccgcgcgaccacctacctcatctcggagcatcgcggagaagtgctggatgccagagcaagacccgtcaaacgccagcggaagggagcagttatagctcaggccgtggtgctgtaccccagcgtactcaaagcagaacgcaaggaagcagaacggagaatcttgctcagcccaccaagtgttctccagtggagacttagcgcaagccatgatgttctcgtggttttcctcaatgaacttgatgcgctcagggaacggaaccttatcgacacccgcacagtttgcaccgtggattttcagccagtagtaaccttccttaccgattggtttacctttcgccagcgtaagcagtcctttggtcatatcgttaccttgcgggttgaacattgacacagcgtaaacacgaccgcgccagtccatgttgtaagggaaccagatggccttatggttagcaaacttattggcttgctcaagcatgaactcaaggctgatacggcgagacttgcgagccttgtccttgcggtacacagcagcggcagcacgtttccacgcggtgagagcctcaggattcatgtcgatgtcttccggtttcatcgggagttcttcacgctcaatcgcagggatgtcctcgaccggacaatgcttccacttggtgattacgttggcgaccgctaggactttcttgttgattttccatgcggtgttttgcgcaatgttaatcgctttgtacacctcaggcatgtaaacgtcttcgtagcgcatcagtgctttcttactgtgagtacgcaccagcgccagaggacgacgaccgttagcccaatagccaccaccagtaatgccagtccacggcttaggaggaactacgcaaggttggaacatcggagagatgccagccagcgcacctgcacgggttgcgatagcctcagcgtattcaggtgcgagttcgatagtctcagagtcttgacctactacgccagcattttggcggtgtaagctaaccattccggttgactcaatgagtcatctcgatgcagcgtactcctacatgaatagagtcttccttatgccacgaagaccacgcctcgccaccgagtagacccttagagagcatgtcagcctcgacaacttgcataaatgctttcttgtagacgtgccctacgcgcttgttgagttgttcctcaacgtttttcttgaagtgcttagcttcaaggtcacggatacgaccgaagcgagcctcgtcctcaatggcccgaccgattgcgcttgctacagcctgaacggttgtattgtcagcactggttaggcaagccagagtggtcttaatggtgatgtacgctacggcttccggcttgatttcttgcaggaactggaaggctgtcgggtgcttgccgcgcttagctttcacttcctcaaaccagtcgttgatgcgtgcaatcatcttagggagtagggtagtgatgagaggcttggcggcagcgttatccgcaacctcaccagctttaagttgacgctcaaacatcttgcggaagcgtgcttcacccatctcgtaagactcatgctcaagggccaactgttcgcgagctaaacgctcaccgtaatggtcagccagagtgttgaacgggatagcagccagttcgatgtcagagaagtcgttcttagcgatgttaatcgtgttcattcctcctcctccactagtttggaccatttgtcatttccccctttgatttaagtgaacaagtttatccatcaactatcttaattgagttagtttgtttatccaataaactaactttatctcatcatatacaaaataaatgtttatttcaatgttttttttagaaaatttagttataatattagatatgatacttttaaatatctaattcaagcttcaaaaaacaccaacttagttcggtggataaacaaaggagtggttattattcaaattgcagatcaagctttagtaaaaaaaatgaatcaaaaattaatattagatgaaattttgaagaactcccctgtctccagggcaactctctctgagattacaggattaaacaagtctactgtctcctctcaagtaaatacactgcttgaaaaagattttatttttgaaattggggcagggcaatctagaggcggcagaagacctgtaatgcttgtttttaataagaatgcaggctactcgattggtattgatataggagtcgactatcttaacggaattctaaccgacttagaaggaaatattattctcgagaagacttctgacttgtctagttcttccgctagtgaagtaaaagagattttatttgcacttattcatggttttgtaacccatatgcctgagtccccttatggtctagtcggaataggaatttgtgttccaggccttgtagatcgtcatcagcaaattattttcatgcctaacttaaattggaatatcaaagatttgcagtttttaattgagagtgagtttaatgttccggtttttgttgaaaatgaagctaatgcaggagcatacggtgaaaaagtatttggtatgacaaaaaactatgaaaacatcgtttacatcagtattaatatcggaattggaactggacttgttattaacaacgaattgtataaaggtgttcagggtttttctggggaaatgggtcatatgacgatagattttaatggacccaaatgcagctgtggaaatcgaggctgttgggaattatatgcttctgaaaaagcgttactggcttcgctctctaaagaagaaaagaatatttctcgaaaagagattgtggaacgcgcaaataaaaatgatgtagaaatgttaaatgcacttcaaaactttggcttttatatcggaattggattaaccaatatccttaatacatttgatatagaagctgttatcttgagaaatcatataattgaatctcatcccattgttttaaatacgattaaaaacgaagtttcttctagagtccattctcatttagacaataaatgtgaactattgccttcttcgttaggaaaaaatgcacctgctttaggagcggtttctatcgttattgattcttttttaagtgttacccctataagttagttaagtgaacgcaaagggcgcctgatgcggtattttctccttacgcatctgtgcggtatttcacaccgcatagatcattatttagcgatgtcgcctactcctgaggacgatccggcaatagttacccttattatcaagataagaaagaaaaggatttttcgctacgctcaaatcctttaaaaaaacacaaaagaccacattttttaatgtggtcttttattcttcaactaaagcacccattagttcaacaaacgaaaattggataaagtgggatatttttaaaatatatatttatgttacagtaatattgacttttaaaaaaggattgattctaatgaagaaagcagacaagtaagcctcctaaattcactttagataaaaatttaggaggcatatcaaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatatgagataatgccgactgtactttttacagtcggttttctaatgtcactaacctgccccgttagttgaagaaggtttttatattacagctccagatctatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcatttattaacgattacctgcttttaattctgcatctaattgcataagctccgccttcattttttccttttcagcttcaaaatcattattcactggaagatcttctgctactgcattttcatcatttgtttgtagccactttggaaccatttctttacgtgcattacgtactacacgtttcctttttgtttgcttcggtttccaatgtttaagacggtattctaatagtgatttatattgcttattcgtatcaacgattacagtatcgttaagcatttcacttttcacttcgtcgaaaggtacattattatatttttctcgtaatttatcatcaataaaatcacgtggactactactaattttttcatttgttaatttttcatttgtttcttttttatttataggtgtacgttctgtgttgtacacactgtgttgtacagaacgtacatcagaagaaatttgcttattttgtatctgatgtacattttgtacatcagatattttttcagtgatttcaagtggcgaatgttgtatcacgcttaacgtcatgacgtttttaccttgcgattgtaagagatttatttgttcaatgacaagcaattcgaaattagcttgcgaaatcggaatatctgatacggcgtaaaaataatctttttttccgtcaacatatgcgctaaaaccgacagcgtattgtttaatagctaattccttccaagcagcgtctacatttctacgagaaaatttgttttgtaactgggtcttcctaatcgtccaaccttcacctaagctcataatatgacttaatagaccgatagaccgcaagtcctccaggtccgtttgtaatggcgcgttatgtatttgagcatattgcgttgtttttattgttttgataattccattcatttctaaattcctccaacgattgattttttgttggagtacttgctataatatacgtaaccaaataaacgtactattgcaacaagtacaaccaagagaacggaggcctcaatctccgttcttttttattatatcatgtcctattgatgaaaataggagtgggtataaaatcggaattttactagagacattgattaatttgcagacagttttagttaacagttgaatataaatgactctagaggatcccgggttacgcgaacgcgaagtccgactctaaga

SEQ ID NO: 29 - DNA, 7171 bp, synthetic construct
  source 1..7171: mol_type = other DNA
tgcagtccggcaaaaaagggcaaggtgtcaattctcatgtttgacagcttatcatcggcaatagttacccttattatcaagataagaaagaaaaggatttttcgctacgctcaaatcctttaaaaaaacacaaaagaccacattttttaatgtggtctttattcttcaactaaagcacccattagttcaacaaacgaaaattggataaagtgggatatttttaaaatatatatttatgttacagtaatattgacttttaaaaaaggattgattctaatgaagaaagcagacaagtaagcctcctaaattcactttagataaaaatttaggaggcatatcaaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatatgagataatgccgactgtactttttacagtcggttttctaatgtcactaacctgccccgttagttgaagaaggtttttatattacagctccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgacatggatgagcgatgatgatatccgtttaggctgggcggtgatagcttctcgttcaggcagtacgcctcttttcttttccagacctgagggaggcggaaatggtgtgaggttcccggggaaaagccaaataggcgatcgcgggagtgctttatttgaagatcaggctatcactgcggtcaatagatttcacaatgtgatggctggacagcctgaggaactctcgaacccgaatggaaacaaccagatatttatgaatcagcgcggctcacatggcgttgtgctggcaaatgcaggttcatcctctgtctctatcaatacggcaacaaaattgcctgatggcaggtatgacaataaagctggagcgggttcatttcaagtgaacgatggtaaactgacaggcacgatcaatgccaggtctgtagctgtgctttatcctgatgatattgcaaaagcgcctcatgttttccttgagaattacaaaacaggtgtaacacattctttcaatgatcaactgacgattaccttgcgtgcagatgcgaatacaacaaaagccgtttatcaaatcaataatggaccagagacggcgtttaaggatggagatcaattcacaatcggaaaaggagatccatttggcaaaacatacaccatcatgttaaaaggaacgaacagtgatggtgtaacgaggaccgagaaatacagttttgttaaaagagatccagcgtcggccaaaaccatcggctatcaaaatccgaatcattggagccaggtaaatgcttatatctataaacatgatgggagccgagtaattgaattgaccggatcttggcctggaaaaccaatgactaaaaatgcagacggaatttacacgctgacgctgcctgcggacacggatacaaccaacgcaaaagtgatttttaataatggcagcgcccaagtgcccggtcagaatcagcctggctttgattacgtgctaaatggtttatataatgactcgggcttaagcggttctcttccccattgagggcaaggctagacgggacttaccgaaagaaaccatcaatgatggtttcttttttgttcataaatcagacaaaacttttctcttgcaaaagtttgtgaagtgttgcacaatataaatgtgaaatacttcacaaacaaaaagacatcaaagagaaacataccctgcaaggatgctgatattgtctgcatttgcgccggagcaaaccaaaaacctggtgagacacgccttgaattagtagaaaagaacttgaagattttcaaaggcatcgttagtgaagtcatggcgagcggatttgacggcattttcttagtcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctactgaagtggatttctttaagagctcctttaacttcctcaccagtagttgtatcggtaccataagtagaagcagcaacccaagtagctttaccagcatccggttcaaccagcatagtaagaatcttactggacatcggcagttcttcgaacagtgcgccaactaccagctctttctccagaatgggctatacctcttttacctaaagagcatgtaactttactggatatagctagaaaaggctatcggggagagtgtgatgataagtgggaaggactatattcaaaggtgaaagcactcgttaagtatatgaaaaattctatagaaacttctctcaattaggctaattttattgcaataacaggtgcttacttttctggagttctttagcaaatttttttattagctgaacttagtattagtggccatactcctccaatccaaagctatttagaaagattactatatcctcaaacaggcggtaaccggcctcttcatcgggaatgcgcgcgaccttcagcatcgccggcatgtccccctggcggacgggaagtatccagctcgaggtcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttgatagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgccggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattaacaaaattctccagtcttcacatcggtttgaaaggaggaagcggaagaatgaagtaagagggatttttgactccgaagtaagtcttcaaaaaatcaaataaggagtgtcaagaatgtttgcaaaacgattcaaaacctctttactgccgttattcgctggatttttattgctgtttcatttggttctggcaggaccggcggctgcgagtgctgaaacggcgaacaaatcgaatgagcttacagcaccgtcgatcaaaagcggaaccattcttcatgcatggaattggtcgttcaatacgttaaaacacaatatgaaggatattcatgatgcaggatatacagccattcagacatctccgattaaccaagtaaaggaagggaatcaaggagataaaagcatgtcgaactggtactggctgtatcagccgacatcgtatcaaattggcaaccgttacttaggtactgaacaagaatttaaagaaatgtgtgcagccgctgaagaatatggcataaaggtcattgttgacgcggtcatcaatcataccaccagtgattatgccgcgatttccaatgaggttaagagtattccaaactggacacatggaaacacacaaattaaaaactggtctgatcggatcctagaagcttatcgaattcgcggccgcttctagagcaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacatactagagaaagaggagaaatactagatggcttcctccgaagacgttatcaaagagttcatgcgtttcaaagttcgtatggaaggttccgttaacggtcacgagttcgaaatcgaaggtgaaggtgaaggtcgtccgtacgaaggtacccagaccgctaaactgaaagttaccaaaggtggtccgctgccgttcgcttgggacatcctgtccccgcagttccagtacggttccaaagcttacgttaaacacccggctgacatcccggactacctgaaactgtccttcccggaaggtttcaaatgggaacgtgttatgaacttcgaagacggtggtgttgttaccgttacccaggactcctccctgcaagacggtgagttcatctacaaagttaaactgcgtggtaccaacttcccgtccgatggtccggttatgcagaaaaaaaccatgggttgggaagcttccaccgaacgtatgtacccggaagacggtgctctgaaaggtgaaatcaaaatgcgtctgaaactgaaagacggtggtcactacgacgctgaagttaaaaccacctacatggctaaaaaaccggttcagctgccgggtgcttacaaaaccgacatcaaactggacatcacctcccacaacgaagactacaccatcgttgaacagtacgaacgtgctgaaggtcgtcactccaccggtgcttaataacgctgatagtgctagtgtagatcgctactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagtagcggccgc

SEQ ID NO: 30 - DNA, 9224 bp, synthetic construct
  source 1..9224: mol_type = other DNA
gggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcagcggccgctactagtatataaacgcagaaaggcccacccgaaggtgagccagtgtgactctagtagagagcgttcaccgacaaacaacagataaaacgaaaggcccagtctttcgactgagcctttcgttttatttgatgcctggctctagtagcgatctacactagcactatcagcgttattaagcaccggtggagtgacgaccttcagcacgttcgtactgttcaacgatggtgtagtcttcgttgtgggaggtgatgtccagtttgatgtcggttttgtaagcacccggcagctgaaccggttttttagccatgtaggtggttttaacttcagcgtcgtagtgaccaccgtctttcagtttcagacgcattttgatttcacctttcagagcaccgtcttccgggtacatacgttcggtggaagcttcccaacccatggtttttttctgcataaccggaccgtcggacgggaagttggtaccacgcagtttaactttgtagatgaactcaccgtcttgcagggaggagtcctgggtaacggtaacaacaccaccgtcttcgaagttcataacacgttcccatttgaaaccttccgggaaggacagtttcaggtagtccgggatgtcagccgggtgtttaacgtaagctttggaaccgtactggaactgcggggacaggatgtcccaagcgaacggcagcggaccacctttggtaactttcagtttagcggtctgggtaccttcgtacggacgaccttcaccttcaccttcgatttcgaactcgtgaccgttaacggaaccttccatacgaactttgaaacgcatgaactctttgataacgtcttcggaggaagccatctagtatttctcctctttctctagtatgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgctctagatttttttttgaattctacagatgcattttatttcatatagtaagtacatcacctattagtttgttgtttaaacaaactaacttattttcatcttatataacctcgtcagtattttcaatattttttttagttttttatgaacacattagatataataaagggaagattcgctatgtactatgttgatacttaatttaaagattaaacaaatggagtggatgaagtggatatcgctgatcaaacctttgtcaaaaaagtaaatcaaaagttattattaaaagaaatccttaaaaattcacctatttcaagagcaaaattatctgaaatgactggattaaataaatcaactgtctcatcacaggtaaacacgttaatgaaagaaagtatggtatttgaaataggtcaaggacaatcaagtggcggaagaagacctgtcatgcttgtttttaataaaaaggcaggatactccgttggaatagatgttggtgtggattatattaatggcattttaacagaccttgaaggaacaatcgttcttgatcaataccgccatttggaatccaattctccagaaataacgaaagacattttgattgatatgattcatcactttattacgcaaatgccccaatctccgtacgggcttattggtataggtatttgcgtgcctggactcattgataaagatcaaaaaattgttttcactccgaactccaactggagagatattgacttaaaatcttcgatacaagagaagtacaatgtgcctgtttttattgaaaatgaggcaaatgctggcgcatatggagaaaaagtatttggagctgcaaaaaatcacgataacattatttacgtaagtatcagcacaggaatagggatcggtgttattatcaacaatcatttatatagaggagtaagcggcttctctggagaaatgggacatatgacaatagactttaatggtcctaaatgcagttgcggaaaccgaggatgctgggaattgtatgcttcagagaaggctttattaaaatctcttcagaccaaagagaaaaaactgtcctatcaagatatcataaacctcgcccatctgaatgatatcggaaccttaaatgcattacaaaattttggattctatttaggaataggccttaccaatattctaaatactttcaacccacaagccgtaattttaagaaatagcataattgaatcgcatcctatggttttaaattcaatgagaagtgaagtatcatcaagggtttattcccaattaggcaatagctatgaattattgccatcttccttaggacagaatgcaccggcattaggaatgtcctccattgtgattgatcattttctggacatgattacaatgtaattttttatggaatggacagctcatctttaaagatgagtttttttattctaggagtatttctgaagcaatagtgacatggcaccttctcatatgaaaaaggagttctaaaatagaaatctcctttttcatgtgcaaattatttttctttataacgaaaatatctaaagtcggccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatccatcctccaaagttggagagtgagttttatgtcgcaaatattaatgtttctggtgaaccttatcaaattttcgttgatttaatagaaacatagcggtaaaattagcagtaacttaatagaacggaaatgaaaaaagccactctcatatgctattggctaccaacctttagcgagaatgacttaatcctgtacagccatacaggacttcgacttataagaggcgccaacttcaaataagttatttgccttgttttcgcgaacaaggcttattagatacacctattgtaccgttactctacgaatatttcaagtagtaattactagcattgtccgttactctacgaatatttcaagtagtaattactagcattgtccgttactctacgaatatttcaagtagtaattactagcattgtcatatacataataaaacggatataaaagggcgttttctatacctagaagtcttgtaaatgtacagggcgtttagatatagagaacgccctttttgtgttccgttccagtggaagctaccactttaaaaagatggtctagtgtagccaatgcaggagagtacactcggatatcagttgtcgttgcattcaactgtctgacgtaagcgaggtaaaggacacaagccttgcataaaacaagcctacgggatgtaaatcctaataatgatgataaccaagacgttagcggcaaaaagtgttgggggttcaaaataagacatgattgtgcgactggagttaaacagttactcgtaagcggcgatcatgacactgattcacggctattcttgtacaagctagctttattacaaggatatgcgggttatatagcgaatcacccgaaagggaacggtgttgggcgtgagaaacgcaccgtacggcgcaatacaatgccaataagctatatacggacggtatagtagttttgtaagctataaccgtttgtcgtcaatgcaaccaatctcaattcgagacctcggcatctaagccagtacgaatgagtgggcgttttaacctcgtaaattttcaacaggggttactatgcccaaaactacattcagatttcctaacaaactcgccagtatgaaaaccttaagaccttaaagtcaagggatttgaaggattttaacctcgattagcaaaaaatgtagagtactgaagcaactaccattaactaagatagtgggggattgaggaagaatccagagctgtttaaatcaagtgaaagacaagatgaaattaaaagaatagtgaaagataggggagtggttctctatgagaaaggaaatggctagagaacaaaggcagcggtttattgatctattgttagactttatggtaaagaatcctcatttatttgttaatggtacagaggatgaaagtaataatgttgttacaaaatgtaatagtgatattaaagaggttgcggagtcatatttaactcttttatagtgagagggttaaaactaattaatatgtattaaggcccaatgttggaattattgtatttcactaggcaacctacttactaaaagtaagattatccattagtggatgttataatattgggttttttaacacaataatcatcgcctttcggtgtcgtttgatagaaaagtaaccattagcgatgaaaaagtcaatataaaaagccatccgtaaaaaacggatggcttaccgtacataggatcgttggtagggcggcgtatcctacatctctggtaacttacctagccaatcaaatgcttgagaacggcggttagataagcgcgtggggaacctttcccacctcaaagatcctatatcattattatgttactttctacaggtagtataccatgttcttatattttagtaaactccccgttagcttaacaggtctttgtaagcaattaaacgtccactattcaatcgtctttggattttcgcaggaccgttttttagatcgaacatagttgataagaacaaataaccgcttgggtccaactttatagcaattagtatatggtcatttaaaatctttaccaattcaacgctattaggttctttaggattttgcccgacatagtcggggtgttcaacgatatcttttatgtgcgatgaatatttttcataaataccaggatgttgtttctttacgtgctttataaatccgggaaacatttttacatcgttagaagtgcaagtcaagttatatgtatctataatgatttgtggaagttttgccacaacagttggtttatttacaatcttttttttattagccgtcaaatttctccctcatctcgtctctttatatctttattttatcataaaggagtatttgaaccgtcgcgcgggacaggtttatgatagggatattttattgaataattgatggtataagggactttcatgcttggaaagtggggattatgaattagatgcttgtccacaatatgttccaatgtaattaaaatttatgttcccaccttgaccaaacatcacgtccatacttaaatcgtccctcctttaataggtaaaatattaatttaccttaataaaaaaataatggataatagtattcgtctgaatttatataatcagggggaactattgatgctggggatactatttacagcggcgccatctactgatgtcgtaaaggatttgcaagataaagttatatcattgcaggatcatgaggtagcgtttttgaacaccacgatatctaatatgttgatccccgaagcaaacttaagagtgtgttgatagtgcagtatcttaaaattttgtgtataataggaattgaagttaaattagatgctaaaaatttgtaattaagaaggagggattcgtcatgttggtattccaaatgcgtaatgtagataaaacatctactgttttgaaacagactaaaaacagtgattacgcagataaataaatacgttagattaattcctaccagtgactaatcttatgactttttaaacagataactaaaattacaaacaaatcgtttaacttctgtatttatttacagatgtaatcacttcaggagtaattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaacattcgtgtcactttaattcaccaagatattctacagtttcaattccctaacaaacagaggtataaaattgttgggagtattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccattacttatgagcaagtattgtctatttttaatagttatctattatttaacgggaggaaataattctatgagtcgcttttttaaatttggaaagttacacgttactaaagggaatggagataaattattagatatactactgacagcttccaagaagctaaagaggtccctagcgcctacggggaatttggggtacattgaaaaaggaagagtatgagtattcaacatttccgtgtcgcccttattcccttttttgcggcattttgccttcctgtttttgctcacccagaaacgctggtgaaagtaaaagatgctgaagatcagtt

SEQ ID NO: 31 - DNA, 707 bp, synthetic construct
  source 1..707: mol_type = other DNA
ttgaattattttgataataaaattgaccagtttgccacgtatcttcaaaagagaaataacttagatcatattcaatttttgcaagtacgattagggatgcaggtcttagctaaaaatataggtaaattaattgttatgtatactattgcctatattttaaacatttttctgtttacgttaattacgaatttaacattttatttaataagaagacatgcacatggtgcacatgcaccttcttctttttggtgttatgtagaaagtattatactatttatacttttacctttagtaatagtaaattttcatattaactttttaattatgattattttaacagttatttctttaggtgtaatctcagtatatgctcctgcagcaactaaaaagaagcccattcctgtgcgacttattaaacgaaaaaaatattatgcgattattgttagtttaacccttttcattatcacacttatcatcaaagagccatttgcccaattcattcaattaggcatcataatagaagctattacattattacctattttctttattaaggaggacttaaaatgaatacattatttaacttattttttgattttattactgggattttaaaaaacattggtaacatcgcagcttatagtacttgtgacttcataatggatgaagttgaagtaccaaaagaattaacacaattacacgaataa

SEQ ID NO: 32 - DNA, 708 bp, synthetic construct
  source 1..708: mol_type = other DNA
ttgaattattttgataacaaaattgaccagtttgccacgtatcttcaaaagagaaataacttagatcatattcaatttttgcaagtacgattagggatgcaggtcttagctaaaaatataggaaaactgattgttatgtatactcttgcctatattttaaacatttttatcttcactttaattactaatatatcgttttatctaattagaaggtatgcacatggtgcacatgcaccttcatctttttggtgttatatagaaagcattacactttttattgttctacctctattagtactacattttcatattaatgaaacactaatgatgtttttagcattattaagtgttggcgtagtgattaaatatgcacctgcagctactaagaagaagcctatcccggcaaggcttgtaaagcaaaaaagatatttttcaattataattagtacaattcttttcattatcacactttttgtaaaggaaccatatacccaatttattcaactgggtattattatacaagctattacattactaccaatatactattcaaaggaggactaaattatgaaaaaattactcaacaaagttattgagcttttagttgactttttcaacagcatcggttacagagcagcctatataaattgtgattttttattggatgaagctgaagtaccaaaagaattaactcaattacacgaataa

SEQ ID NO: 33 - DNA, 149 bp, synthetic construct
  source 1..149: mol_type = other DNA
ctagtatcattcattctattataaaggaaaagcttgcgaattgatacgtgcggaagggaaggacttgaccgcaaatccttccccgttccgcttattcatttgccgcttttgtctggtctgatttttgctctagaagcggccgcgaattc

SEQ ID NO: 34 - DNA, 169 bp, synthetic construct
  source 1..169: mol_type = other DNA
ctagagagtcaatgtatgaatggatacgggatatgaatcaataagtacgtgaaagagaaaagcaacccagatatgatagggaacttttctctttcttgttttacattgaatctttacaatcctattgatataatctaagctagtgtattttgcgtttaatagttactag

SEQ ID NO: 35 - DNA, 249 bp, synthetic construct
  source 1..249: mol_type = other DNA
ctagagggagttctgagaattggtatgccttataagtccaattaacagttgaaaacctgcataggagagctatgcgggttttttattttacataatgatacataatttaccgaaacttgcggaacataattgaggaatcatagaattttgtcaaaataattttattgacaacgtcttattaacgttgatataatttaaattttatttgacaaaaatgggctcgtgttgtacaataaatgtagttactag

SEQ ID NO: 36 - DNA, 744 bp, synthetic construct
  source 1..744: mol_type = other DNA
atgacaagattatgggcatcattgctaactgttattatttatatattgtctcaatttttaccgcttctcattgtaaaaaaattaccatttgtacaatatagtggcatagaactgactaaagcagtcatttacatacaacttgttctatttttaatcgccgccacgacgattattttaattaatttaaaaattaaaaatccaacaaaattagaattagaagttaaagaacctaaaaaatatatcattccatgggcattgcttggatttgcattggtaatgatttatcaaatggtagtgagcattgtattaacgcaaatttatggtggacaacaagtaagtcctaatacagaaaagctaattattattgctcgaaaaatacctatatttatcttctttgtatctattattggtcctttattagaagaatatgtattcagaaaagtaatctttggagaattatttaatgcgattaaaggtaatcgtatcgtggcatttattattgctacaacagtaagttcattaatatttgcattagcacataatgatttcaaatttattccagtttattttggtatgggtgtcattttttcattagcatatgtttggacaaaacggcttgctgttccaattattatccatatgttacaaaacggatttgtcgttatattccaattacttaatcctgaggctttgaaaaaagccacggaacaagcgaattttatatatcacatttttattccataa

SEQ ID NO: 37 - DNA, 570 bp, synthetic construct
  source 1..570: mol_type = other DNA
ttgaattattttgataataaaattgaccagtttgccacgtatcttcaaaagagaaataacttagatcatattcaatttttgcaagtacgattagggatgcaggtcttagctaaaaatataggtaaattaattgttatgtatactattgcctatattttaaacatttttctgtttacgttaattacgaatttaacattttatttaataagaagacatgcacatggtgcacatgcaccttcttctttttggtgttatgtagaaagtattatactatttatacttttacctttagtaatagtaaattttcatattaactttttaattatgattattttaacagttatttctttaggtgtaatctcagtatatgctcctgcagcaactaaaaagaagcccattcctgtgcgacttattaaacgaaaaaaatattatgcgattattgttagtttaacccttttcattatcacacttatcatcaaagagccatttgcccaattcattcaattaggcatcataatagaagctattacattattacctattttctttattaaggaggacttaaaatga

SEQ ID NO: 38 - DNA, 563 bp, synthetic construct
  source 1..563: mol_type = other DNA
ttgaattattttgataacaaaattgaccagtttgccacgtatcttcaaaagagaaataacttagatcatattcaatttttgcaagtacgattagggatgcaggtcttagctaaaaatataggaaaactgattgttatgtatactcttgcctatattttaaacatttttatcttcactttaattactaatatatcgttttatctaattagaaggtatgcacatggtgcacatgcaccttcatctttttggtgttatatagaaagcattacactttttattgttctacctctattagtactacattttcatattaatgaaacactaatgatgtttttagcattattaagtgttggcgtagtgattaaatatgcacctgcagctactaagaagaagcctatcccggcaaggcttgtaaagcaaaaaagatatttttcaattataattagtacaattcttttcattatcacactttttgtaaaggaaccatatacccaatttattcaactgggtattattatacaagctattacattactaccaatatactattcaaaggaggacta

SEQ ID NO: 39 - DNA, 141 bp, synthetic construct
  source 1..141: mol_type = other DNA
atgaatacattatttaacttattttttgattttattactgggattttaaaaaacattggtaacatcgcagcttatagtacttgtgacttcataatggatgaagttgaagtaccaaaagaattaacacaattacacgaataa

SEQ ID NO: 40 - DNA, 141 bp, synthetic construct
  source 1..141: mol_type = other DNA
atgaaaaaattactcaacaaagttattgagcttttagttgactttttcaacagcatcggttacagagcagcctatataaattgtgattttttattggatgaagctgaagtaccaaaagaattaactcaattacacgaataa

SEQ ID NO: 41 - DNA, 141 bp, synthetic construct
  source 1..141: mol_type = other DNA
atgaatacattatttaacttattttttgattttattactgggattttaaaaaacattggtaacatcgcagcttatagtacttgtgccttcataatggatgaagttgaagtaccaaaagaattaacacaattacacgaataa

SEQ ID NO: 42 - DNA, 141 bp, synthetic construct
  source 1..141: mol_type = other DNA
atgaaaaaattactcaacaaagttattgagcttttagttgactttttcaacagcatcggttacagagcagcctatataaattgtgcttttttattggatgaagctgaagtaccaaaagaattaactcaattacacgaataa

SEQ ID NO: 43 - DNA, 29 bp, synthetic construct
  source 1..29: mol_type = other DNA
gatacgcacccattagggtgcgaaaaaaa